(19) United States
(12) Patent Application Publication
(10) Pub. No.: US 2024/0041896 A1
(43) Pub. Date: Feb. 8, 2024

(54) CERTAIN (2S)-N-[(1S)-1-CYANO-2-PHENYLETHYL]-1,4-OXAZEPANE-2-CARBOXAMIDES FOR TREATING BRONCHIECTASIS

(71) Applicants: AstraZeneca AB, Södertälje (SE); Insmed Incorporated, Bridgewater, NJ (US)

(72) Inventors: Hans Roland LONN, Cheshire (GB); Stephen CONNOLLY, Cheshire (GB); Steven SWALLOW, Cheshire (GB); Reinilde HEYRMAN, Bridgewater, NJ (US); Eugene SULLIVAN, Bridgewater, NJ (US); Carlos FERNANDEZ, Bridgewater, NJ (US)

(73) Assignees: AstraZeneca AB, Södertälje (SE); Insmed Incorporated, Bridgewater, NJ (US)

(21) Appl. No.: 18/450,379

(22) Filed: Aug. 15, 2023

Related U.S. Application Data
(63) Continuation of application No. 17/576,470, filed on Jan. 14, 2022, now abandoned.
Application No. 17/576,470 is a continuation of application No. 16/511,726, filed on Jul. 15, 2019, now abandoned.
Application No. 16/511,726 is a continuation of application No. 16/198,068, filed on Nov. 21, 2018, now abandoned.
Application No. 16/198,068 is a continuation of application No. 15/662,709, filed on Jul. 28, 2017, now abandoned.
(60) Provisional application No. 62/368,400, filed on Jul. 29, 2016.

(51) Int. Cl. A61K 31/553; A61K 9/00

(52) U.S. Cl. CPC A61K 31/553; A61K 9/0053

(57) ABSTRACT

The present disclosure relates to methods for treating bronchiectasis, for example, non-cystic fibrosis bronchiectasis with compositions comprising an effective amount of certain (2S)—N-[(1S)-1-cyano-2-phenylethyl]-1,4-oxazepane-2-carboxamide compounds of Formula (I), including pharmaceutically acceptable salts thereof,

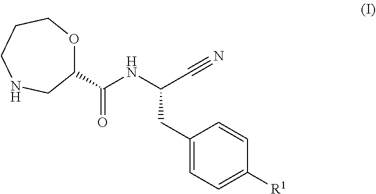

that inhibit dipeptidyl peptidase 1 (DPP1) activity. Methods provided herein are useful for prophylaxis, increasing the lung function in a patient, and/or and/or decreasing the rate of pulmonary exacerbation in a patient. In one embodiment, the compound of Formula (I) is (2S)—N-{(1S)-1-cyano-2-[4-(3-methyl-2-oxo-2,3-dihydro-1,3-benzoxazol-5-yl)phenyl]ethyl}-1,4-oxazepane-2-carboxamide.

### CROSS REFERENCE TO RELATED APPLICATION

This application is a continuation of U.S. application Ser. No. 17/576,470, filed Jan. 14, 2022, which is a continuation of U.S. application Ser. No. 16/511,726, filed Jul. 15, 2019, which is a continuation of U.S. application Ser. No. 16/198,068, filed Nov. 21, 2018, which is a continuation of U.S. application Ser. No. 15/662,709, filed Jul. 28, 2017, which claims priority from U.S. Provisional Application Ser. No. 62/368,400, filed Jul. 29, 2016, the disclosure of each of which is incorporated by reference herein in its entirety for all purposes.

### BACKGROUND OF THE INVENTION

Bronchiectasis is a disease characterized by localized, irreversible enlargement of bronchi and bronchioles that may lead to obstructed breathing caused by abnormal mucus production. Bronchiectasis symptoms typically include a chronic dry or wet cough. Other symptoms include shortness of breath, coughing up blood, and chest pain. Wheezing and nail clubbing may also occur. People with the disease often get frequent lung infections.

Bronchiectasis, along with chronic obstructive pulmonary disease (COPD), acute lung injury, acute respiratory distress syndrome, and cystic fibrosis (CF) are all conditions of severe pulmonary dysfunction resulting from a massive inflammatory response. The histological characteristic of these inflammatory lung diseases is the accumulation of neutrophils in the interstitium and alveoli of the lung. Neutrophil activation leads to the release of multiple cytotoxic products including reactive oxygen species and proteases (serine, cysteine, and metalloproteases).

Subjects having bronchiectasis experience pulmonary exacerbations with an average frequency ranging from 1.5 to 6 per year (Goeminne et al. Respir Med. 2014; 108(2):287-96; Kelly et al. Eur J Intern Med 2003; 14(8):488-92; Chalmers et al. Am J Respir Crit Care Med. 2014; 189(5):576-85). Currently, there is no standard-of-care (SOC) pharmacological treatment bronchiectasis. The primary goal of treatment is to treat underlying cause, prevent disease progression, maintain or improve lung function, and improve the symptoms and quality of life.

The present invention addresses the need for a therapy effective for the treatment of bronchiectasis, e.g., in non-cystic fibrosis patients.

### SUMMARY OF THE INVENTION

In one aspect, a method for treating a bronchiectasis patient is provided. The method comprises, in one embodiment, administering to a patient in need thereof a pharmaceutical composition comprising an effective amount of a compound of formula (I), or a pharmaceutically acceptable salt of a compound of formula (I):

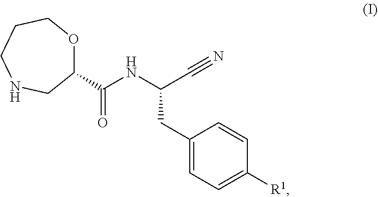

wherein,
R¹ is

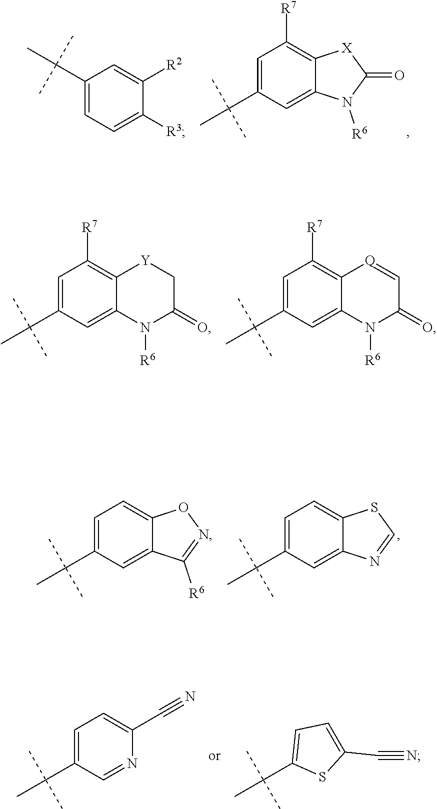

R² is hydrogen, F, Cl, Br, OSO₂C₁₋₃alkyl, or C₁₋₃alkyl;
R³ is hydrogen, F, Cl, Br, CN, CF₃, SO₂C₁₋₃alkyl, CONH₂ or SO₂NR⁴R⁵, wherein R⁴ and R⁵ together with the nitrogen atom to which they are attached form an azetidine, pyrrolidine or piperidine ring;
R⁶ is C₁₋₃alkyl, optionally substituted by 1, 2 or 3 F and/or optionally by OH, OC₁₋₃alkyl, N(C₁₋₃alkyl)₂, cyclopropyl, or tetrahydropyran;
R⁷ is hydrogen, F, Cl or CH₃;
X is O, S or CF₂;
Y is O or S; and
Q is CH or N.

The bronchiectasis patient in one embodiment, is present in a cystic fibrosis patient. In another embodiment, the patient treated with one of the methods provided herein does not have cystic fibrosis (referred to herein as “non-CF bronchiectasis”).

In one embodiment of the method for treating bronchiectasis in a patient in need thereof, the pharmaceutical composition comprises an effective amount of (2S)—N-{(1S)-1-cyano-2-[4-(3-methyl-2-oxo-2,3-dihydro-1,3-benzoxazol-5-yl)phenyl]ethyl}-1,4-oxazepane-2-carboxamide,

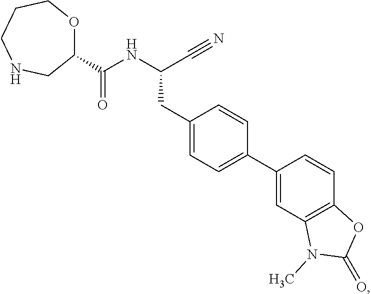 [SMILES: Cn1c(=O)oc2ccc(-c3ccc(C[C@@H](C#N)NC(=O)[C@@H]4CNCCCO4)cc3)cc21]

or a pharmaceutically acceptable salt thereof.

In one embodiment of the method, the patient is administered the composition once daily. In another embodiment, the patient is administered the composition twice daily, or every other day, or once a week. Administration, in one embodiment, is via the oral route.

In one embodiment of the method for treating bronchiectasis, the treating comprises increasing the length of time to first pulmonary exacerbation, as compared to an untreated bronchiectasis patient. In a further embodiment, the increasing comprises increasing by about 1 day, about 3 days, about 1 week, about 2 weeks, about 3 weeks, about 4 weeks, about 5 weeks or about 6 weeks, or increasing by at least about 1 day, at least about 3 days, at least about 1 week, at least about 2 weeks, at least about 3 weeks, at least about 4 weeks, at least about 5 weeks or at least about 6 weeks. In another embodiment, the increasing comprising increasing of from about 20 days to about 100 days, or from about 30 days to about 100 days, or from about 20 days to about 75 days, or from about 20 days to about 50 days, or from about 20 days to about 40 days.

In another embodiment of a method for treating bronchiectasis, a patient in need of treatment is administered a composition comprising an effective amount of a compound of formula (I), or a pharmaceutically acceptable salt thereof. The treating comprises reducing the rate of pulmonary exacerbation in the patient, as compared to the rate of pulmonary exacerbation experienced by the patient prior to treatment, or compared to an untreated bronchiectasis patient. In a further embodiment, the rate is calculated over a period of about 1 week, about 1 month, about 2 months, about 3 months, about 4 months, about 5 months, about 6 months, about 9 months, about 12 months, about 15 months, about 18 months, about 21 months or about 24 months. In a further embodiment, the rate of pulmonary exacerbation in the patient is reduced by about 15%, by about 20%, by about 25%, by about 30%, by about 35%, by about 40% or by about 50%, by about 55%, by about 60%, by about 65%, by about 70%, by at least about 5%, by at least about 10%, by at least about 15%, by at least about 20%, at least about 25%, at least about 30%, at least about 35%, at least about 40%, or at least about 50%, at least about 70% as compared to the rate of pulmonary exacerbation experienced by the patient prior to treatment, or compared to an untreated bronchiectasis patient.

In another embodiment of a method for treating bronchiectasis, a patient in need of treatment is administered a composition comprising an effective amount of a compound of formula (I), or a pharmaceutically acceptable salt thereof. In this embodiment, treating comprises reducing the duration of a pulmonary exacerbation in the patient, as compared to the duration of a pulmonary exacerbation experienced by the patient prior to treatment, or compared to an untreated bronchiectasis patient. In a further embodiment, the reduced duration of a pulmonary exacerbation is a reduced duration of about 12 hours, about 24 hours, about 48 hours or about 72 hours, at least about 6 hours, at least about 12 hours, at least about 24 hours, at least about 48 hours, at least about 72 hours, at least about 96 hours, at least about 120 hours, at least about 144 hours or at least about 168 hours. In another embodiment, the reduced duration of a pulmonary exacerbation is a reduced duration of about 6 hrs to about 96 hrs, about 12 hrs to about 96 hrs, about 24 hrs to about 96 hrs, about 48 hrs to about 96 hrs or about 48 hrs to about 168 hrs. In yet another embodiment, the reduced duration of a pulmonary exacerbation is a reduced duration of about 1 day to about 1 week, about 2 days to about 1 week, about 3 days to about 1 week, about 4 days to about 1 week, about 5 days to about 1 week or about 6 days to about 1 week. In yet another embodiment, the reduced duration of a pulmonary exacerbation is a reduced duration of about 1 day to about 2 weeks, about 2 days to about 2 weeks, about 4 days to about 2 weeks, about 6 days to about 2 weeks, about 8 days to about 2 weeks or about 10 days to about 2 weeks.

In another embodiment of a method for treating bronchiectasis, a patient in need of treatment is administered a composition comprising an effective amount of a compound of formula (I), or a pharmaceutically acceptable salt thereof. In this embodiment, the treating comprises improving the lung function of the patient, as compared to the lung function of the patient prior to treatment, or as compared to an untreated bronchiectasis patient.

In one embodiment, the improvement in lung function is an increase in forced expiratory volume in one second (FEV₁), as compared to the FEV₁ of the patient prior to treatment, or as compared to an untreated bronchiectasis patient. In a further embodiment, the increase in FEV₁ is an increase by about 5%, about 10%, about 15%, by about 20%, by about 25%, by about 30%, by about 35%, by about 40%, by about 45% or by about 50%. In another embodiment, the increase in FEV₁ is an increase by at least about 5%, at least about 10%, at least about 15%, by at least about 20%, by at least about 25%, by at least about 30%, by at least about 35%, by at least about 40%, by at least about 45% or by at least about 50%. In yet another embodiment, the increase in FEV₁ is an increase by about 5% to about 50%, by about 5% to about 40%, by about 5% to about 30%, by about 5% to about 20%, by about 10% to about 50%, by about 15% to about 50%, by about 20% to about 50% or by about 25% to about 50%. In even another embodiment, the increase in FEV₁ is an increase of about 25 mL to about 500 mL, or about 25 mL to about 250 mL.

In another embodiment, the improvement in lung function in the patient is an increase in forced vital capacity (FVC), as compared to the lung function of the patient prior to treatment, or as compared to an untreated bronchiectasis patient. In a further embodiment, the increase in FVC is an increase by about 1%, increase by about 2%, by about 3%, by about 4%, by about 5%, by about 6%, by about 7%, by about 8%, by about 9%, by about 10%, by about 11%, by about 12%, by about 13%, by about 14%, by about 15%, by about 16%, by about 17%, by about 18%, by about 19%, by about 20%, by about 25%, by about 30%, by about 35%, by about 40%, by about 45%, by about 50%, by about 55%, by about 60%, by about 65%, by about 70%, by about 75%, by about 80%, by about 85% or by about 90%, as compared to a FVC of the patient prior to treatment, or as compared to an untreated bronchiectasis patient.

In another embodiment of a method for treating bronchiectasis, a patient in need of treatment is administered a composition comprising an effective amount of a compound of formula (I), or a pharmaceutically acceptable salt thereof. The treating comprises improving the patient's quality of life (QOL), as compared to the patient's QOL prior to treatment. The QOL is assessed by the Leicester Cough Questionnaire (LCQ), by the St. George's Respiratory Questionnaire (SGRQ), or the Quality of Life-Bronchiectasis (QOL-B) questionnaire.

In yet another embodiment of a method for treating bronchiectasis, a patient in need of treatment is administered a composition comprising an effective amount of a compound of formula (I), or a pharmaceutically acceptable salt thereof. In this embodiment, the treating comprises decreasing active neutrophil elastase (NE) sputum concentration in the patient, as compared to the active NE sputum concentration prior to treatment. In a further embodiment, decreasing the active NE sputum concentration comprises decreasing by about 1%, about 5%, about 10%, about 20%, about 25%, about 30%, at least about 1%, at least about 5%, at least about 10%, at least about 20%, at least about 25%, at least about 30%, at least about 40%, at least about 50%, at least about 60%, or at least about 70%. In another embodiment, a patient treated via one of the methods provided herein has a lower NE sputum concentration as compared to an untreated patient. In a further embodiment, the active NE sputum concentration is about 1%, about 5%, about 10%, about 20%, about 25%, about 30%, at least about 1%, at least about 5%, at least about 10%, at least about 20%, at least about 25%, at least about 30%, at least about 40%, at least about 50%, at least about 60%, or at least about 70% lower than the active NE concentration of the untreated patient.

In even another embodiment of a method for treating bronchiectasis, a patient in need of treatment is administered a composition comprising an effective amount of a compound of formula (I), or a pharmaceutically acceptable salt thereof. In this embodiment, the treating comprises lightening the patient's sputum color as compared to the patient's sputum color prior to treatment, as measured by the sputum color chart of Murray. In a further embodiment, lightening the patient's sputum color comprises lightening the patient's sputum color by a single gradation. In a further embodiment, the lightening is from purulent (dark yellow and/or dark green) to mucopurulent (pale yellow and/or pale green). In another embodiment, the lightening is from mucopurulent (pale yellow and/or pale green) to mucoid (clear). In yet another embodiment, the lightening is from purulent (dark yellow and/or dark green) to mucoid (clear).

### DETAILED DESCRIPTION OF THE INVENTION

Neutrophils contain four main types of granules: (i) azurophilic or primary granules, (ii) specific or secondary granules, (iii) gelatinase or tertiary granules, and (iv) secretory granules. Azurophilic granules are believed to be the first to form during neutrophil maturation in the bone marrow and are characterized by the expression of related neutrophil serine proteases (NSPs): neutrophil elastase (NE), proteinase 3, and cathepsin G. The lysosomal cysteine dipeptidyl peptidase 1 (DPP1) is the proteinase that activates these 3 NSPs by removal of the N-terminal dipeptide sequences from their precursors during azurophilic granule assembly (Pham et al. (2004). J Immunol. 173(12), pp. 7277-7281). DPP1 is broadly expressed in tissues, but is highly expressed in cells of hematopoietic lineage such as neutrophils.

The three NSPs, abundantly secreted into the extracellular environment upon neutrophil activation at inflammatory sights, are thought to act in combination with reactive oxygen species to assist in degradation of engulfed microorganisms inside phagolysosomes. A fraction of the released proteases remains bound in an active form on the external surface of the plasma membrane so that both soluble and membrane-bound NSPs can regulate the activities of a variety of biomolecules such as chemokines, cytokines, growth factors, and cell surface receptors. Regulation is thought to occur by either converting the respective biomolecule to an active form or by degrading the biomolecule by proteolytic cleavage. Secreted proteases can stimulate mucus secretion and inhibit mucociliary clearance, but also activate lymphocytes and cleave apoptotic and adhesion molecules (Bank and Ansorge (2001). J Leukoc Biol. 69, pp. 197-206; Pham (2006). Nat Rev Immunol. 6, pp. 541-550; Meyer-Hoffert (2009). Front Biosci. 14, pp. 3409-3418; Voynow et al. (2004). Am J Physiol Lung Cell Mol Physiol. 287, pp. L1293-302; the disclosure of each of which is incorporated by reference in its entirety for all purposes).

The physiological balance between proteases and anti-proteases is required for the maintenance of the lung's connective tissue. For example, an imbalance in favor of proteases can result in lung injury (Umeki et al. (1988). Am J Med Sci. 296, pp. 103-106; Tetley (1993). Thorax 48, pp. 560-565; the disclosure of each of which is incorporated by reference in its entirety for all purposes).

The methods provided herein employ reversible inhibitors of DPP1. Without wishing to be bound by theory, it is thought that the compounds of formula (I), administered via the methods provided herein have beneficial effects via decreasing inflammation and mucus hypersecretion, which in turn leads to a decrease in pulmonary exacerbations, a decrease in the rate of pulmonary exacerbations, and/or an improvement in cough, sputum production, and/or lung function (e.g., forced expiratory volume in 1 second [FEV₁]) in bronchiectasis patients. Without wishing to be bound by theory, it is thought that the methods provided herein modify bronchiectasis progression by reducing the accelerated rate of lung function decline and/or lung tissue destruction.

It is to be understood that where in this specification a group is qualified by “defined above” the said group encompasses the first occurring and broadest definition as well as each and all the other definitions for that group.

As used herein, “C₁₋₃” means a carbon group having 1, 2 or 3 carbon atoms.

The term “alkyl”, unless otherwise noted, includes both straight and branched chain alkyl groups and may be, substituted or non-substituted. “Alkyl” groups include, but are not limited to, methyl, ethyl, n-propyl, i-propyl, butyl, pentyl.

The the term “pharmaceutically acceptable”, unless otherwised noted, is used to characterize a moiety (e.g., a salt, dosage form, or excipient) as being appropriate for use in accordance with sound medical judgment. In general, a pharmaceutically acceptable moiety has one or more benefits that outweigh any deleterious effect that the moiety may have. Deleterious effects may include, for example, excessive toxicity, irritation, allergic response, and other problems and complications.

Provided herein are methods for treating bronchiectasis patients via administration of a pharmaceutical composition comprising an effective amount of a compound of formula (I), or a pharmaceutically acceptable salt thereof:

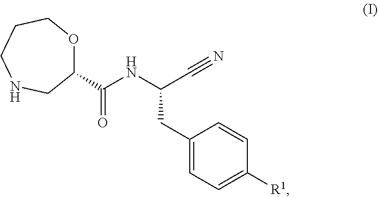

wherein,
R¹ is

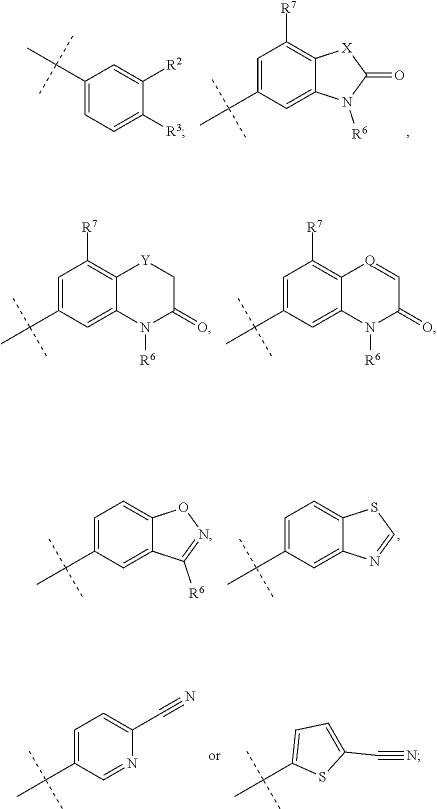

R² is hydrogen, F, Cl, Br, OSO₂C₁₋₃alkyl, or C₁₋₃alkyl;
R³ is hydrogen, F, Cl, Br, CN, CF₃, SO₂C₁₋₃alkyl, CONH₂ or SO₂NR⁴R⁵, wherein R⁴ and R⁵ together with the nitrogen atom to which they are attached form an azetidine, pyrrolidine or piperidine ring;
R⁶ is C₁₋₃alkyl, optionally substituted by 1, 2 or 3 F and/or optionally by OH, OC₁₋₃alkyl, N(C₁₋₃alkyl)₂, cyclopropyl, or tetrahydropyran;
R⁷ is hydrogen, F, Cl or CH₃;
X is O, S or CF₂;
Y is O or S; and
Q is CH or N.

The bronchiectasis can be present in a cystic fibrosis patient. In another embodiment, the bronchiectasis is not associated with cystic fibrosis (non-CF bronchiectasis).

In one embodiment R¹ is

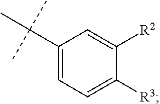

R² is hydrogen, F, Cl, Br, OSO₂C₁₋₃alkyl, or C₁₋₃alkyl; R³ is hydrogen, F, Cl, Br, CN, CF₃, SO₂C₁₋₃alkyl, CONH₂ or SO₂NR⁴R⁵, wherein R⁴ and R⁵ together with the nitrogen atom to which they are attached form an azetidine, pyrrolidine or piperidine ring.

In a further embodiment, R¹ is

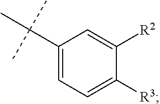

R² is hydrogen, F, Cl or C₁₋₃alkyl; and R³ is hydrogen, F, Cl, CN or SO₂C₁₋₃alkyl.

In still a further embodiment, R¹ is

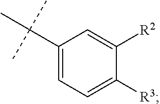

R² is hydrogen, F or C₁₋₃alkyl; and R³ is hydrogen, F or CN.

In another embodiment, R¹ is

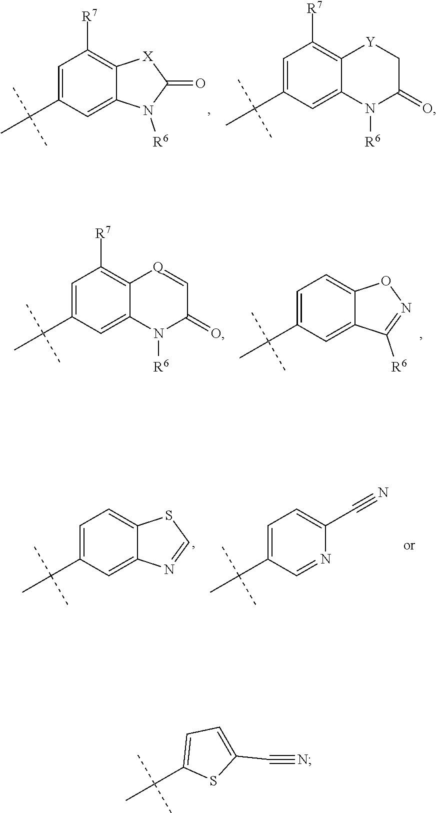

X is O, S or CF₂; Y is O or S; Q is CH or N; R⁶ is C₁₋₃alkyl, wherein the C₁₋₃alkyl is optionally substituted by 1, 2 or 3 F and/or optionally substituted by OH, OC₁₋₃alkyl, N(C₁₋₃alkyl)₂, cyclopropyl, or tetrahydropyran; and R⁷ is hydrogen, F, Cl or CH₃.

In still a further embodiment, R¹ is

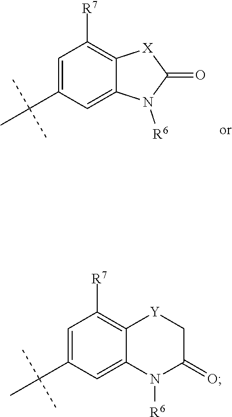

X is O, S or CF₂; Y is O or S; R⁶ is C₁₋₃alkyl, optionally substituted by 1, 2 or 3 F and optionally substituted by OH, OC₁₋₃alkyl, N(C₁₋₃alkyl)₂, cyclopropyl, or tetrahydropyran; and R⁷ is hydrogen, F, Cl or CH₃.

In still a further embodiment, R¹ is

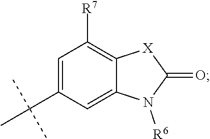

X is O, S or CF₂; R⁶ is C₁₋₃alkyl, wherein the C₁₋₃alkyl is optionally substituted by 1, 2 or 3 F; and R⁷ is hydrogen, F, Cl or CH₃.

In still a further embodiment, R¹ is

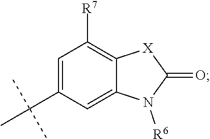

X is O; R⁶ is C₁₋₃alkyl, wherein the C₁₋₃alkyl is optionally substituted by 1, 2 or 3 F; and R⁷ is hydrogen.

In one embodiment, R² is hydrogen, F, Cl, Br, OSO₂C₁₋₃alkyl or C₁₋₃alkyl.

In a further embodiment, R² is hydrogen, F, Cl or C₁₋₃alkyl.

In still a further embodiment, R² is hydrogen, F or C₁₋₃alkyl.

In one embodiment, R³ is hydrogen, F, Cl, Br, CN, CF₃, SO₂C₁₋₃alkyl CONH₂ or SO₂NR⁴R⁵, wherein R⁴ and R⁵ together with the nitrogen atom to which they are attached form an azetidine, pyrrolidine or piperidine ring.

In a further embodiment, R³ is selected from hydrogen, F, Cl, CN or SO₂C₁₋₃alkyl.

In still a further embodiment, R³ is selected from hydrogen, F or CN.

In one embodiment, R⁶ is C₁₋₃alkyl, wherein said C₁₋₃alkyl is optionally substituted by 1, 2 or 3 F and optionally by one substituent selected from OH, OC₁₋₃alkyl, N(C₁₋₃alkyl)₂, cyclopropyl, or tetrahydropyran.

In a further embodiment, R⁶ is C₁₋₃alkyl, wherein said C₁₋₃alkyl is optionally substituted by 1, 2 or 3 F. In still a further embodiment, R⁶ is methyl or ethyl. In still a further embodiment, R⁶ is methyl.

In one embodiment, R⁷ is hydrogen, F, Cl or CH₃. In a further embodiment R⁷ is hydrogen.

In one embodiment, the compound of Formula (I) is (2S)—N-{(1S)-1-cyano-2-[4-(3-methyl-2-oxo-2,3-dihydro-1,3-benzoxazol-5-yl)phenyl]ethyl}-1,4-oxazepane-2-carboxamide:

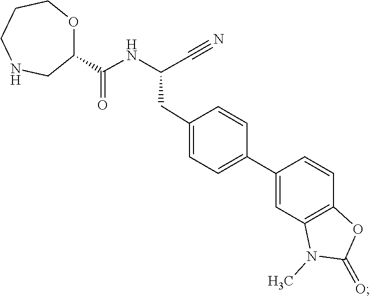 [SMILES: Cn1c(=O)oc2ccc(-c3ccc(C[C@@H](C#N)NC(=O)[C@@H]4CNCCCO4)cc3)cc21]

or a pharmaceutically acceptable salt thereof.

In one embodiment, the compound of formula (I) is:
(2S)—N-[(1S)-1-Cyano-2-(4′-cyanobiphenyl-4-yl)ethyl]-1,4-oxazepane-2-carboxamide,
(2S)—N-{(1S)-1-Cyano-2-[4-(3-methyl-2-oxo-2,3-dihydro-1,3-benzoxazol-5-yl)phenyl]ethyl}-1,4-oxazepane-2-carboxamide,
(2S)—N-{(1S)-1-Cyano-2-[4-(3,7-dimethyl-2-oxo-2,3-dihydro-1,3-benzoxazol-5-yl)phenyl]ethyl}-1,4-oxazepane-2-carboxamide,
4′-[(2S)-2-Cyano-2-{[(2S)-1,4-oxazepan-2-ylcarbonyl]amino}ethyl]biphenyl-3-yl methanesulfonate,
(2S)—N-{(1S)-1-Cyano-2-[4-(3-methyl-1,2-benzoxazol-5-yl)phenyl]ethyl}-1,4-oxazepane-2-carboxamide,
(2S)—N-{(1S)-1-Cyano-2-[4′-(trifluoromethyl)biphenyl-4-yl]ethyl}-1,4-oxazepane-2-carboxamide,
(2S)—N-[(1S)-1-Cyano-2-(3′,4′-difluorobiphenyl-4-yl)ethyl]-1,4-oxazepane-2-carboxamide,
(2S)—N-{(1S)-1-Cyano-2-[4-(6-cyanopyridin-3-yl)phenyl]ethyl}-1,4-oxazepane-2-carboxamide,
(2S)—N-{(1S)-1-Cyano-2-[4-(4-methyl-3-oxo-3,4-dihydro-2H-1,4-benzothiazin-6-yl)phenyl]ethyl}-1,4-oxazepane-2-carboxamide,
(2S)—N-{(1S)-1-Cyano-2-[4-(3-ethyl-7-methyl-2-oxo-2,3-dihydro-1,3-benzoxazol-5-yl)phenyl]ethyl}-1,4-oxazepane-2-carboxamide,
(2S)—N-[(1S)-1-Cyano-2-{4-[3-(2-hydroxy-2-methylpropyl)-2-oxo-2,3-dihydro-1,3-benzoxazol-5-yl]phenyl}ethyl]-1,4-oxazepane-2-carboxamide,
(2S)—N-[(1S)-1-Cyano-2-{4-[3-(2,2-difluoroethyl}-7-fluoro-2-oxo-2,3-dihydro-1,3-benzoxazol-5-yl]phenyl}ethyl]-1,4-oxazepane-2-carboxamide,
(2S)—N-[(1S)-1-Cyano-2-(4-{3-[2-(dimethylamino)ethyl]-2-oxo-2,3-dihydro-1,3-benzoxazol-5-yl}phenyl)ethyl]-1,4-oxazepane-2-carboxamide,
(2S)—N-{(1S)-1-Cyano-2-[4-(3,3-difluoro-1-methyl-2-oxo-2,3-dihydro-1H-indol-6-yl)phenyl]ethyl}-1,4-oxazepane-2-carboxamide,
(2S)—N-{(1S)-1-Cyano-2-[4-(7-fluoro-3-methyl-2-oxo-2,3-dihydro-1,3-benzoxazol-5-yl)phenyl]ethyl}-1,4-oxazepane-2-carboxamide,
(2S)—N-{(1S)-1-Cyano-2-[4-(3-ethyl-2-oxo-2,3-dihydro-1,3-benzoxazol-5-yl)phenyl]ethyl}-1,4-oxazepane-2-carboxamide,
(2S)—N-[(1S)-1-Cyano-2-{4-[3-(cyclopropylmethyl)-2-oxo-2,3-dihydro-1,3-benzoxazol-5-yl]phenyl}ethyl]-1,4-oxazepane-2-carboxamide,
(2S)—N-[(1S)-1-Cyano-2-{4-[3-(2-methoxyethyl)-2-oxo-2,3-dihydro-1,3-benzothiazol-5-yl]phenyl}ethyl]-1,4-oxazepane-2-carboxamide,
(2S)—N-[(1S)-1-Cyano-2-{4-[2-oxo-3-(propan-2-yl)-2,3-dihydro-1,3-benzoxazol-5-yl]phenyl}ethyl]-1,4-oxazepane-2-carboxamide,
(2S)—N-{(1S)-1-Cyano-2-[4-(4-methyl-3-oxo-3,4-dihydro-2H-1,4-benzoxazin-6-yl)phenyl]ethyl}-1,4-oxazepane-2-carboxamide,
(2S)—N-[(1S)-1-Cyano-2-{4-[3-(2-methoxyethyl)-2-oxo-2,3-dihydro-1,3-benzoxazol-5-yl]phenyl}ethyl]-1,4-oxazepane-2-carboxamide,
(2S)—N-{(1S)-1-Cyano-2-[4-(5-cyanothiophen-2-yl)phenyl]ethyl}-1,4-oxazepane-2-carboxamide,
(2S)—N-[(1S)-2-(4′-Carbamoyl-3′-fluorobiphenyl-4-yl)-1-cyanoethyl]-1,4-oxazepane-2-carboxamide,
(2S)—N-{(1S)-1-Cyano-2-[4-(1-methyl-2-oxo-1,2-dihydroquinolin-7-yl)phenyl]ethyl}-1,4-oxazepane-2-carboxamide,
(2S)—N-[(1S)-1-Cyano-2-{4-[2-oxo-3-(tetrahydro-2H-pyran-4-ylmethyl)-2,3-dihydro-1,3-benzoxazol-5-yl]phenyl}ethyl]-1,4-oxazepane-2-carboxamide,
(2S)—N-{(1S)-2-[4-(7-Chloro-3-methyl-2-oxo-2,3-dihydro-1,3-benzoxazol-5-yl)phenyl]-1-cyanoethyl}-1,4-oxazepane-2-carboxamide,
(2S)—N-[(1S)-1-Cyano-2-{4-[3-(2,2-difluoroethyl}-2-oxo-2,3-dihydro-1,3-benzoxazol-5-yl]phenyl}ethyl]-1,4-oxazepane-2-carboxamide,
(2S)—N-[(1S)-1-Cyano-2-{4-[2-oxo-3-(2,2,2-trifluoroethyl)-2,3-dihydro-1,3-benzoxazol-5-yl]phenyl}ethyl]-1,4-oxazepane-2-carboxamide,
(2S)—N-{(1S)-1-Cyano-2-[4-(3-methyl-2-oxo-2,3-dihydro-1,3-benzothiazol-5-yl)phenyl]ethyl}-1,4-oxazepane-2-carboxamide,
(2S)—N-{(1S)-1-Cyano-2-[4′-(methylsulfonyl)biphenyl-4-yl]ethyl}-1,4-oxazepane-2-carboxamide,
(2S)—N-{(1S)-2-[4′-(Azetidin-1-ylsulfonyl)biphenyl-4-yl]-1-cyanoethyl}-1,4-oxazepane-2-carboxamide,
(2S)—N-[(1S)-1-Cyano-2-(4′-fluorobiphenyl-4-yl)ethyl]-1,4-oxazepane-2-carboxamide,
(2S)—N-{(1S)-2-[4-(1,3-Benzothiazol-5-yl)phenyl]-1-cyanoethyl}-1,4-oxazepane-2-carboxamide, or
(2S)—N-[(1S)-1-Cyano-2-(4′-cyanobiphenyl-4-yl)ethyl]-1,4-oxazepane-2-carboxamide,

or a pharmaceutically acceptable salt of one of the foregoing compounds.

The methods provided herein comprise the administration of a composition comprising an effective amount of a compound of formula (I), or a pharmaceutically acceptable salt thereof, to a bronchiectasis patient in need of treatment. The compounds of formula (I) and their pharmaceutically acceptable salts are inhibitors of dipeptidyl peptidase 1 (DPP1) activity. The bronchiectasis may be in a patient with cystic fibrosis, or a patient that does not have cystic fibrosis (sometimes referred to as “bronchiectasis unrelated to cystic fibrosis” or “non-CF bronchiectasis”). Administration routes include oral administration. Administration schedules can be determined by the user of the method, e.g., a prescribing physician. In one embodiment, administration is once daily. In another embodiment, administration is twice daily. In another embodiment, administration is every other day, 3× per week or 4× per week.

Non-CF bronchiectasis has been reported to be caused by or associated with numerous aetiologies ranging from genetic illness to retained airway foreign body, and has been reported to be present in patients with systemic disease, common respiratory diseases such as chronic obstructive pulmonary disease (COPD) as well as uncommon diseases such as sarcoidosis (Chang and Bilton (2008). Thorax 63, pp. 269-276, incorporated by reference herein in its entirety for all purposes).

Bronchiectasis is considered a pathological endpoint that results from many disease processes and is a persistent or progressive condition characterized by dilated thick-walled bronchi. The symptoms vary from intermittent episodes of expectoration and infection localized to the region of the lung that is affected to persistent daily expectoration often of large volumes of purulent sputum. Bronchiectasis may be associated with other non-specific respiratory symptoms. The underlying pathological process of bronchiectasis, without wishing to be bound by theory, has been reported as damage to the airways which results from an event or series of events where inflammation is central to the process (Guideline for non-CF Bronchiectasis, Thorax, July 2010, V. 65(Suppl 1), incorporated by reference herein in its entirety for all purposes).

The term “treating” in one embodiment, includes: (1) preventing or delaying the appearance of clinical symptoms of the state, disorder or condition developing in the patient that may be afflicted with or predisposed to the state, disorder or condition but does not yet experience or display clinical or subclinical symptoms of the state, disorder or condition; (2) inhibiting the state, disorder or condition (i.e., arresting, reducing or delaying the development of the disease, or a relapse thereof in case of maintenance treatment, of at least one clinical or subclinical symptom thereof); (3) relieving the condition (i.e., causing regression of the state, disorder or condition or at least one of its clinical or subclinical symptoms). In one embodiment, the clinical symptom is a pulmonary exacerbation and/or (4) prophylaxis of the bronchiectasis, e.g., non-CF bronchiectasis.

Prophylaxis is expected to be particularly relevant to the treatment of persons who have suffered a previous episode of, or are otherwise considered to be at increased risk of, bronchiectasis. As such, in one embodiment, of the invention, a method for providing prophylaxis of bronchiectasis in a patient in need thereof is provided. The patient in need thereof, in one embodiment, has suffered a previous episode of, or is at increased risk for being diagnosed with bronchiectasis. The method comprises administering a composition comprising an effective amount of a compound of Formula (I) or a pharmaceutically acceptable salt thereof to the patient. In a further embodiment, the compound of Formula (I) is (2S)—N-{(1S)-1-cyano-2-[4-(3-methyl-2-oxo-2,3-dihydro-1,3-benzoxazol-5-yl)phenyl]ethyl}-1,4-oxazepane-2-carboxamide, or a pharmaceutically acceptable salt thereof. Without wishing to be bound by theory, it is thought that administration of an effective amount of a compound of Formula (I), or a pharmaceutically acceptable salt thereof, interrupts the cycle of infection/inflammation/impaired mucociliaryclearance and tissue destruction observed in bronchiectasis patients by inhibiting neutrophil elastase activity.

A “pulmonary exacerbation” as used herein, is three or more of the following symptoms exhibited for at least 48 hours by a patient: (1) increased cough; (2) increased sputum volume or change in sputum consistency; (3) increased sputum purulence; (4) increased breathlessness and/or decreased exercise tolerance; (5) fatigue and/or malaise; (6) hemoptysis. In one embodiment, the three or more symptoms result in a physician's decision to prescribe an antibiotic(s) to the patient exhibiting the symptoms.

In one embodiment, the treating via administering a composition comprising an effective amount of a compound of formula (I) comprises increasing the length of time to pulmonary exacerbation, as compared to the length of time to pulmonary exacerbation in an untreated bronchiectasis patient. For example, in some embodiments, the length of time to pulmonary exacerbation is increased at least about 20 days, as compared to the length of time to pulmonary exacerbation in an untreated bronchiectasis patient. In other embodiments, the length of time to pulmonary exacerbation is increased from about 20 to about 100 days, as compared to the length of time to pulmonary exacerbation in an untreated bronchiectasis patient. In another embodiment, the length of time to pulmonary exacerbation is increased from about 25 to about 100 days, from about 30 to about 100 days, from about 35 to about 100 days or from about 40 to about 100 days, as compared to the length of time to pulmonary exacerbation in an untreated bronchiectasis patient. In other embodiments, the increase is from about 25 to about 75 days, from about 30 to about 75 days, from about 35 to about 75 days or from about 40 to about 75 days, as compared to the length of time to pulmonary exacerbation in an untreated bronchiectasis patient. In other embodiments, the increase in time to pulmonary exacerbation is about 30 to about 60 days, as compared to the length of time to pulmonary exacerbation in an untreated bronchiectasis patient. In a further embodiment, the compound of formula (I) is (2S)—N-{(1S)-1-cyano-2-[4-(3-methyl-2-oxo-2,3-dihydro-1,3-benzoxazol-5-yl)phenyl]ethyl}-1,4-oxazepane-2-carboxamide, or a pharmaceutically acceptable salt thereof.

In one embodiment, the increasing of the time between pulmonary exacerbation comprises increasing by about 1 day, about 3 days, about 1 week, about 2 weeks, about 3 weeks, about 4 weeks, about 5 weeks or about 6 weeks, or increasing by at least about 1 day, at least about 3 days, at least about 1 week, at least about 2 weeks, at least about 3 weeks, at least about 4 weeks, at least about 5 weeks or at least about 6 weeks. In another embodiment, the increasing comprising increasing of from about 20 days to about 100 days, or from about 30 days to about 100 days, or from about 20 days to about 75 days, or from about 20 days to about 50 days, or from about 20 days to about 40 days. In a further embodiment, the compound of formula (I) is (2S)—N-{(1S)-1-cyano-2-[4-(3-methyl-2-oxo-2,3-dihydro-1,3-benzoxazol-5-yl)phenyl]ethyl}-1,4-oxazepane-2-carboxamide, or a pharmaceutically acceptable salt thereof.

In yet another embodiment, a method for treating bronchiectasis, e.g., non-CF bronchiectasis, is provided comprising administering a composition comprising an effective amount of a compound of formula (I), or a pharmaceutically acceptable salt thereof, to a patient in need thereof. In one embodiment, the compound is administered orally, once daily. Treating comprises reducing the rate of pulmonary exacerbation, as compared to the rate of pulmonary exacerbation experienced by the patient prior to treatment, or as compared to an untreated bronchiectasis patient. The rate of pulmonary exacerbations can be calculated by dividing the number of exacerbations by a specific time period, e.g., 1 day, 1 week, about 1 month, about 2 months, about 3 months, about 4 months, about 5 months, about 6 months, about 9 months, about 12 months, about 15 months, about 18 months, about 21 months or about 24 months. The reduction in rate of exacerbations, in one embodiment, is a reduction by about 15%, by about 20%, by about 25%, by about 30%, by about 35%, by about 40% or by about 50%, by about 55%, by about 60%, by about 65%, by about 70%, by at least about 5%, by at least about 10%, by at least about 15%, by at least about 20%, at least about 25%, at least about 30%, at least about 35%, at least about 40%, or at least about 50%, at least about 70% as compared to the rate of pulmonary exacerbation experienced by the patient prior to treatment, or compared to an untreated bronchiectasis patient.

In another embodiment, the reduction in rate of exacerbations, in one embodiment, is a reduction by at least about 5%, by at least about 10%, by at least about 15%, by at least about 20%, at least about 25%, at least about 30%, at least about 35%, at least about 40%, or at least about 50%. In one embodiment, the compound of formula (I) is (2S)—N-{(1S)-1-cyano-2-[4-(3-methyl-2-oxo-2,3-dihydro-1,3-benzoxazol-5-yl)phenyl]ethyl}-1,4-oxazepane-2-carboxamide, or a pharmaceutically acceptable salt thereof.

In even another embodiment, a method for treating bronchiectasis, e.g., non-CF bronchiectasis is provided comprising administering to a patient in need thereof, a composition comprising an effective amount of a compound of formula (I), or a pharmaceutically acceptable salt thereof. In one embodiment, the compound is administered orally, once daily. The method comprises decreasing the duration of pulmonary exacerbation, as compared to the duration of a pulmonary exacerbation experienced by the patient prior to treatment, or as compared to an untreated bronchiectasis patient. The reduced duration of a pulmonary exacerbation is a reduced duration of about 12 hours, about 24 hours, about 48 hours or about 72 hours, at least about 6 hours, at least about 12 hours, at least about 24 hours, at least about 48 hours, at least about 72 hours, at least about 96 hours, at least about 120 hours, at least about 144 hours or at least about 168 hours. In another embodiment, the reduced duration of a pulmonary exacerbation is a reduced duration of about 6 hrs to about 96 hrs, about 12 hrs to about 96 hrs, about 24 hrs to about 96 hrs, about 48 hrs to about 96 hrs or about 48 hrs to about 168 hrs. In yet another embodiment, the reduced duration of a pulmonary exacerbation is a reduced duration of about 1 day to about 1 week, about 2 days to about 1 week, about 3 days to about 1 week, about 4 days to about 1 week, about 5 days to about 1 week or about 6 days to about 1 week. In yet another embodiment, the reduced duration of a pulmonary exacerbation is a reduced duration of about 1 day to about 2 weeks, about 2 days to about 2 weeks, about 4 days to about 2 weeks, about 6 days to about 2 weeks, about 8 days to about 2 weeks or about 10 days to about 2 weeks.

The reduced duration, in another embodiment, is a reduction by about 6 hrs to about 96 hrs, about 12 hrs to about 96 hrs, about 24 hrs to about 96 hrs, about 48 hrs to about 96 hrs or about 48 hrs to about 168 hrs.

The reduced duration in one embodiment is the average reduction of exacerbations experienced during treatment. In a further embodiment, the compound of formula (I) is (2S)—N-{(1S)-1-cyano-2-[4-(3-methyl-2-oxo-2,3-dihydro-1,3-benzoxazol-5-yl)phenyl]ethyl}-1,4-oxazepane-2-carboxamide, or a pharmaceutically acceptable salt thereof.

In another embodiment, a method for treating bronchiectasis, e.g., non-CF bronchiectasis, is provided comprising administering a compound of formula (I) to a patient in need thereof. In one embodiment, the compound is administered orally, once daily. In this embodiment, treating comprises reducing the number of pulmonary exacerbation-related hospitilizations of the patient as compared to the number of pulmonary exacerbation-related hospitilzations of the patient prior to treatment, or as compared to an untreated bronchiectasis patient. The number of hospitilzations in one embodiment, is measured over the treatment period and compared to the same length of time prior to treatment or in an untreated bronchiectasis patient. In a further embodiment, the compound of formula (I) is (2S)—N-{(1S)-1-cyano-2-[4-(3-methyl-2-oxo-2,3-dihydro-1,3-benzoxazol-5-yl)phenyl]ethyl}-1,4-oxazepane-2-carboxamide, or a pharmaceutically acceptable salt thereof.

In one embodiment of the methods provided herein, a method for treating bronchiectasis, e.g., non-CF bronchiectasis is provided comprising administering a composition comprising an effective amount of a compound of formula (I), or a pharmaceutically acceptable salt thereof, to a patient in need thereof, wherein the method comprises increasing the lung function in the patient, as compared to the lung function in the patient prior to treatment, or as compared to an untreated bronchiectasis patient. The compound of formula (I) in one embodiment, is (2S)—N-{(1S)-1-cyano-2-[4-(3-methyl-2-oxo-2,3-dihydro-1,3-benzoxazol-5-yl)phenyl]ethyl}-1,4-oxazepane-2-carboxamide, or a pharmaceutically acceptable salt thereof.

The increase in lung function in one embodiment, is measured by spirometry.

Increasing lung function, in one embodiment, comprises increasing the post-bronchodilator forced expiratory volume in 1 second (FEV₁), increasing the forced vital capacity (FVC), increasing the peak expiratory flow rate (PEFR), or increasing the forced expiratory flow of the FVC between 25% and 75% (FEF25-75), as compared to the respective value prior to treatment, or as compared to an untreated bronchiectasis patient. Increasing, in one embodiment, is by about 5%, about 10%, about 15%, by about 20%, by about 25%, by about 30%, by about 35%, by about 40%, by about 45% or by about 50% of the respective value. Increasing, in one embodiment, is by at least about 5%, at least about 10%, at least about 15%, by at least about 20%, by at least about 25%, by at least about 30%, by at least about 35%, by at least about 40%, by at least about 45% or by at least about 50%. In yet another embodiment, the increase is by about 5% to about 50%, by about 5% to about 40%, by about 5% to about 30% or by about 5% to about 20%. In even another embodiment, increasing is by about 10% to about 50%, by about 15% to about 50%, by about 20% to about 50% or by about 25% to about 50%.

The assessment of lung function, e.g, via FEV₁, PEFR or FEF₂₅₋₇₅ measurement, in one embodiment, comprises comparing the lung function in the patient prior to treatment, e.g., immediately prior to treatment, to a time point during treatment, to an average of measurements taken during treatment, or after treatment has completed.

As provided herein, treatment via a method of the invention, in one embodiment, comprises improving the lung function in the patient, wherein the lung function is measured by spirometry. Spirometry is a physiological test that measures how an individual inhales or exhales volumes of air. The primary signal measured in spirometry may be volume or flow. For the methods described herein, pulmonary function test (PFT) by spirometry (e.g., FEV₁, FVC, PEFR, and FEF₂₅₋₇₅) is performed per the American Thorasic Society (ATS)/European Respiratory Society (ERS) criteria, e.g., as set forth by Miller et al. (Miller et al. (2005). Standardization of Spirometry. Eur. Respir. J. 26, pp. 319-38, incorporated by reference herein in its entirety for all purposes).

In one embodiment, the spirometer is capable of accumulating volume for greater than or equal to 15 seconds, e.g., ≥20 seconds, ≥25 seconds, 30 seconds, ≥35 seconds. The spirometer in one embodiment can measure volumes of ≥8 L (BTPS) with an accuracy of at least ±3% of reading or ±0.050 L, whichever is greater, with flows between 0 and 14 L·s⁻¹. In one embodiment, the total resistance to airflow of the spirometer at 14 L·s⁻¹ is <1.5 cmH₂O·L⁻¹·s⁻¹ (0.15 kPa? L⁻¹·s⁻¹). In one embodiment, the total resistance of the spirometer is measured with any tubing, valves, pre-filter, etc. included that may be inserted between the patient and the spirometer. With respect to devices that exhibit changes in resistance due to water vapor condensation, in one embodiment, spirometer accuracy requirements are met under BTPS (body temperature, ambient pressure, saturated with water vapor) conditions for up to eight successive FVC maneuvers performed in a 10-min period without inspiration from the instrument.

With respect to the forced expiratory maneuvers described herein, in one embodiment, the range and accuracy recommendations as set forth in Table 6 of Miller et al. are met (Miller et al. (2005). Standardization of Spirometry. Eur. Respir. J. 26, pp. 319-38, incorporated by reference herein in its entirety for all purposes).

In one embodiment, the improvement in lung function is an improvement in the forced vital capacity (FVC), i.e., the maximal volume of air exhaled with maximally forced effort from a maximal inspiration. This measurement is expressed in liters at body temperature and ambient pressure saturated with water vapor (BTPS).

“Forced vital capacity” (FVC) denotes the volume of gas which is exhaled during a forced expiration starting from a position of full inspiration and ending at complete expiration and is one measure of treatment efficacy. In one embodiment of the methods provided herein, improving the patient's lung function comprises improving the patient's FVC, compared to the patient's FVC prior to treatment, or compared to an untreated bronchiectasis patient. In one embodiment, the FVC of a treated patient is greater by about 1%, greater by about 2%, greater by about 3%, greater by about 4%, greater by about 5%, greater by about 6%, greater by about 7%, greater by about 8%, greater by about 9%, greater by about 10%, greater by about 11%, greater by about 12%, greater by about 13%, greater by about 14%, greater by about 15%, greater by about 16%, greater by about 17%, greater by about 18%, greater by about 19%, greater by about 20%, greater by about 25%, greater by about 30%, greater by about 35%, greater by about 40%, greater by about 45%, greater by about 50%, greater by about 55%, greater by about 60%, greater by about 65%, greater by about 70%, greater by about 75%, greater by about 80%, greater by about 85% or greater by about 90%, as compared to a FVC of the patient prior to treatment, or as compared to an untreated bronchiectasis patient.

FVC maneuvers can be performed according to the procedures known to those of ordinary skill in the art. Briefly, the three distinct phases to the FVC manuever are (1) maximal inspiration; (2) a “blast” of exhalation and (3) continued complete exhalation to the end of test (EOT). The maneuver can be carried out via the closed circuit method or open circuit method. In either instance, the subject inhales rapidly and completely with a pause of less than 1 second at total lung capacity (TLC). The subject then exhales maximally until no more air can be expelled while maintaining an upright posture. The exhalation begins with a “blast” of air from the lungs and then is encouraged to fully exhale. Enthusiastic coaching of the subject continues for a minimum of three manuevers.

The improvement in lung function, in one embodiment, is an improvement compared to lung function immediately prior to treatment, or compared to an untreated bronchiectasis patient. In a further embodiment, improving lung function comprises increasing the forced expiratory volume in one second (FEV₁) of the patient compared to the patient's FEV₁ prior to treatment, or compared to an untreated bronchiectasis patient's FEV₁. FEV is the volume of gas exhaled in a specified time (typically 1 second, i.e., FEV₁) from the start of the forced vital capacity maneuver (Quanjer et al. (1993). Eur. Respir. J. 6, Suppl. 16, pp. 5-40, incorporated by reference herein in its entirety for all purposes).

The increase in FEV₁, in one embodiment, is an increase of at least about 5%, for example, from about 5% to about 50%, or about 10% to about 50%, or about 15% to about 50%. In another embodiment, the FEV₁ of the treated patient is greater by about 1%, greater by about 2%, greater by about 3%, greater by about 4%, greater by about 5%, greater by about 6%, greater by about 7%, greater by about 8%, greater by about 9%, greater by about 10%, greater by about 11%, greater by about 12%, greater by about 13%, greater by about 14%, greater by about 15%, greater by about 16%, greater by about 17%, greater by about 18%, greater by about 19%, greater by about 20%, greater by about 25%, greater by about 30%, greater by about 35%, greater by about 40%, greater by about 45%, greater by about 50%, greater by about 55%, greater by about 60%, greater by about 65%, greater by about 70%, greater by about 75%, greater by about 80%, greater by about 85%, or greater by about 90%, compared to a FEV₁ of the patient prior to treatment, or compared to an untreated bronchiectasis patient.

In another embodiment, the improving lung function comprises increasing the patient's FEV₁ by about 25 mL to about 500 mL, or about 25 mL to about 250 mL, or about 50 mL to about 200 mL, as compared to a FEV₁ of the patient prior to treatment, or as compared to an untreated bronchiectasis patient.

In one embodiment, improving lung function comprises improving the mean forced expiratory flow between 25% and 75% of the FVC (FEF₂₅₋₇₅) (also referred to as the maximum mid-expiratory flow) of the patient, as compared to a FEF₂₅₋₇₅ of the patient prior to treatment, or as compared to an untreated bronchiectasis patient. The measurement is dependent on the validity of the FVC measurement and the level of expiratory effort. The FEF₂₅₋₇₅ index is taken from the blow with the largest sum of FEV₁ and FVC.

In one embodiment, improving lung function comprises improving the peak expiratory flow rate (PEFR) of the patient. The improvement is an improvement compared to PEFR immediately prior to treatment, or as compared to an untreated bronchiectasis patient. The PEFR measures the fastest rate of air that can be expired by a subject. In one embodiment, the PEFR of a treated patient is greater by about 1%, greater by about 2%, greater by about 3%, greater by about 4%, greater by about 5%, greater by about 6%, greater by about 7%, greater by about 8%, greater by about 9%, greater by about 10%, greater by about 11%, greater by about 12%, greater by about 13%, greater by about 14%, greater by about 15%, greater by about 16%, greater by about 17%, greater by about 18%, greater by about 19%, greater by about 20%, greater by about 25%, greater by about 30%, greater by about 35%, greater by about 40%, greater by about 45%, greater by about 50%, greater by about 55%, greater by about 60%, greater by about 65%, greater by about 70%, greater by about 75%, greater by about 80%, greater by about 85% or greater by about 90%, as compared to a PEFR of the patient prior to treatment, or as compared to an untreated bronchiectasis patient.

In yet another embodiment of the invention, a method for treating bronchiectasis is provided comprising administering a composition comprising an effective amount of a compound of formula (I) to a patient in need thereof, wherein treating comprising increasing the quality of life (QOL) of the patient, as compared to the quality of life of the patient prior to treatment, e.g., a baseline value. The compound of formula (I) in one embodiment, is (2S)—N-{(1S)-1-cyano-2-[4-(3-methyl-2-oxo-2,3-dihydro-1,3-benzoxazol-5-yl)phenyl]ethyl}-1,4-oxazepane-2-carboxamide, or a pharmaceutically acceptable salt thereof.

In one embodiment, the QOL of the patient is assessed via the Quality of Life-Bronchiectasis (QOL-B) questionnaire. The QOL-B questionnaire is a validated, self-administered Patient Reported Outcome (PRO) that assesses symptoms, functioning and health-related QOL for subjects with bronchiectasis (Quittner et al. (2014). Chest 146(2), pp. 437-448; Quittner et al. (2015) Thorax 70(1), pp. 12-20, each of which is incorporated by reference in its entirety for all purposes). The QOL-B contains 37 items on 8 domains (Respiratory Symptoms, Physical Functioning, Role Functioning, Emotional Functioning, Social Functioning, Vitality, Health Perceptions and Treatment Burden).

In another embodiment, the QOL of the patient is assessed via the Leicester Cough Questionnaire (LCQ). An improvement in QOL in one embodiment, is a change from baseline (prior to treatment) in LCQ score for the patient. The LCQ is a validated questionnaire evaluating cough on QOL in subjects with bronchiectasis and other conditions where cough is a common symptom (Murray et al. (2009). Eur Respir J. 34: 125-131, incorporated by reference herein in its entirety for all purposes). The LCQ comprises 19 items and takes 5 to 10 minutes to complete. Each item assesses symptoms or the impact of symptoms over the last 2 weeks on a seven-point Likert scale. Scores in three domains (physical, psychological and social) are calculated as a mean for each domain (range 1 to 7). A total score (range 3 to 21) is also calculated by adding the domain scores together. Higher scores indicate better QOL.

In another embodiment, the QOL of the patient is assessed via the St. George's Respiratory Questionnaire (SGRQ). An improvement in QOL in one embodiment, is a change from baseline (prior to treatment) in SGRQ score for the patient. The St. George's Respiratory Questionnaire (SGRQ) is self-administered with 50 questions designed to measure and quantify health-related health status in subjects with chronic airflow limitation (Jones et al. (1991). Respir Med. 85 Suppl B 25-31; discussion 33-7, incorporated by reference herein in its entirety for all purposes). The SGRQ assesses health related quality of life by evaluating 3 health domains: (1) symptoms (distress caused by respiratory symptoms), (2) activity (effects of disturbances to mobility and physical activity), and (3) impact (the effect of disease on factors such as employment, personal control of one's health, and need for medication). It has been shown to correlate well with the established measures of the 3 domains in subjects with asthma and COPD. It has also been validated for use in NCFBE. A composite total score is derived as the sum of domain scores for symptoms, activity, and impact with 0 the best possible score and 100 the worst possible score. A reduction in score of 4 units is generally recognized as a clinically meaningful improvement in QOL.

In another embodiment of the method for treating bronchiectasis provided herein, a composition comprising an effective amount of a compound of formula (I), or a pharmaceutically acceptable salt thereof, is administered to a patient in need thereof, wherein the method comprises decreasing active neutrophil elastase (NE) sputum concentration, as compared to the patient's NE sputum concentration, prior to treatment. In one embodiment, the compound of formula (I) is administered via oral administration. In a further embodiment, administration is 1× daily, every other day, 2× weekly, 3× weekly or 4× weekly. The compound of formula (I) in one embodiment, is (2S)—N-{(1S)-1-cyano-2-[4-(3-methyl-2-oxo-2,3-dihydro-1,3-benzoxazol-5-yl)phenyl]ethyl}-1,4-oxazepane-2-carboxamide, or a pharmaceutically acceptable salt thereof.

Decreasing active NE sputum concentration, in one embodiment, comprises decreasing by about 10%, about 20%, about 25%, about 30%, about 40%, about 50%, about 60%, about 70%, about 80%. In another embodiment, decreasing active NE sputum concentration comprises decreasing by at least about 1%, at least about 5%, at least about 10%, at least about 20%, at least about 30%, at least about 40%, at least about 50%, at least about 60%, at least about 70% or at least about 80%.

In even another embodiment of the method for treating bronchiectasis provided herein, an effective amount of a compound of formula (I) is administered to a patient in need thereof, wherein the method comprises lightening the sputum color of the patient, as measured by the sputum color chart of Murray 2009 (Murray et al. (2009). Eur Respir J. 2009; 34:361-364, incorporated by reference herein in its entirety for all purposes), as compared to the patient's sputum color, prior to treatment. In one embodiment, the compound of formula (I) is administered via oral administration. In a further embodiment, administration is 1× daily, every other day, 2× weekly, 3× weekly or 4× weekly. The compound of formula (I) in one embodiment, is (2S)—N-{(1S)-1-cyano-2-[4-(3-methyl-2-oxo-2,3-dihydro-1,3-benzoxazol-5-yl)phenyl]ethyl}-1,4-oxazepane-2-carboxamide, or a pharmaceutically acceptable salt thereof.

The lightening of color, in one embodiment, is a lightening by a single gradation. For example, in one embodiment, the lightening is from purulent (dark yellow and/or dark green) to mucopurulent (pale yellow and/or pale green). In another embodiment, the lightening is from mucopurulent (pale yellow and/or pale green) to mucoid (clear).

The change in color, in another embodiment, is a lightening of two gradations, i.e., the lightening is from purulent (dark yellow and/or dark green) to mucoid (clear).

Sputum induction is carried out if the patient cannot produce sputum on his or her own. Sputum induction, in one embodiment, is initiated via patient nebulization of a saline solution. The percentage of saline, e.g., 3% or 7% or 10% or 13%, is decided based on the user of the method's preference. The selected saline is placed in the nebulizer, and the subject is in a sitting up or in a semi-fowler position. The subject in one embodiment, wears a nose clip during the nebulization. The subject breathes slowly and deeply through the nebulizer mouthpiece inhaling the salt water mist. The subject is reminded to not breathe quickly but to have slow, deep breaths pausing at peak inspiration to allow deposition of particles. The nebulization time in one embodiment, is 10 minutes.

At the end of nebulization, the subject is instructed to take a few deep breaths, swallow the extra saliva in his/her mouth and attempt to cough up a sputum sample. The subject is encouraged to cough forcefully using the deep coughing method and/or “huffing” cough method. All sputum is deposited in the specimen container. The procedure can be repeated if the amount of sputum collected, e.g., less than 1 mL, less than 2 mL, or less than 3 mL, is not sufficient.

The methods provided herein can be utilized to treat a bronchiectasis patient (e.g., a non-CF bronchiectasis patient) that presents with a pulmonary infection. In one embodiment, the pulmonary infection is a mycobacterial infection. The mycobacterial infection can be a Mycobacterium tuberculosis infection or a non-tuberculous mycobacterium (NTM). Examples of NTM infections that a patient treatable by the methods provided herein can present with include, but are not limited to, M. avium, M. avium subsp. hominissuis (MAH), M. abscessus, M. chelonae, M. bolletii, M. kansasii, M. ulcerans, M. avium, M. avium complex (MAC) (M. avium and M. intracellulare), M. conspicuum, M. kansasih, M. peregrinum, M. immunogenum, M. xenopi, M. marinum, M. malmoense, M. marinum, M. mucogenicum, M. nonchromogenicum, M. scrofulaceum, M. simiae, M. smegmatis, M. szulgai, M. terrae, M. terrae complex, M. haemophilum, M. genavense, M. asiaticum, M. shimoidei, M. gordonae, M. nonchromogenicum, M. triplex, M. lentiflavum, M. celatum, M. fortuitum, M. fortuitum complex (M. fortuitum and M. chelonae) or a combination thereof.

Other pulmonary infections that a bronchiectasis patient can present with include, but are not limited to, Haemophilus influenzae, Pseudomonas aeruginosa, Streptococcus pneumoniae, Staphylococcus aureus and Moraxella catarrhalis. In a further embodiment, the pulmonary bacterial infection is a Pseudomonas aeruginosa infection.

A compound of formula (I), or a pharmaceutically acceptable salt thereof, may also be administered in conjunction with other compounds used for the treatment of bronchiectasis via one of the methods described herein.

The second active ingredient is administered concurrently, sequentially or in admixture with a compound of Formula (I), for the treatment of bronchiectasis, e.g., non-CF bronchiectasis.

The second active ingredient, in one embodiment, is a glucocorticoid receptor agonist (steroidal or non-steroidal) such as triamcinolone, triamcinolone acetonide, prednisone, mometasone furoate, loteprednol etabonate, fluticasone propionate, fluticasone furoate, fluocinolone acetonide, dexamethasone cipecilate, desisobutyryl ciclesonide, clobetasol propionate, ciclesonide, butixocort propionate, budesonide, beclomethasone dipropionate, alclometasone dipropionate, 2,2,2-trifluoro-N-[(1S,2R)-2-[1-(4-fluorophenyl)indazol-5-yl]oxy-2-(3-methoxyphenyl)-1-methyl-ethyl]acetamide, or 3-[5-[(1R,2S)-2-(2,2-difluoropropanoylamino)-1-(2,3-dihydro-1,4-benzodioxin-6-yl)propoxy]indazol-1-yl]-N-[(3R)-tetrahydrofuran-3-yl]benzamide.

The second active ingredient, in another embodiment, is a p38 antagonist such as PH797804 (3-[3-Bromo-4-(2,4-difluoro-benzyloxy)-6-methyl-2-oxo-2H-pyridin-1-yl]-4,N-dimethyl-benzamide), losmapimod, PF03715455 (1-[5-tert-butyl-2-(3-chloro-4-hydroxy-phenyl)pyrazol-3-yl]-3-[[2-[[3-[2-(2-hydroxyethylsulfanyl)phenyl]-[1,2,4]triazolo[4,3-a]pyridin-6-yl]sulfanyl]phenyl]methyl]urea) or N-cyclopropyl-3-fluoro-4-methyl-5-[3-[[1-[2-[2-(methylamino)ethoxy]phenyl]cyclopropyl]amino]-2-oxo-pyrazin-1-yl]benzamide.

The second active ingredient, in yet another embodiment, is a phosphodiesterase (PDE) inhibitor such as a methylxanthanine including theophylline and aminophylline or a selective PDE isoenzyme inhibitor (including a PDE4 inhibitor or an inhibitor of the isoform PDE4D) such as tetomilast, roflumilast, oglemilast, ibudilast, GPD-1116 (3-benzyl-5-phenyl-1H-pyrazolo[4,3-c][1,8]naphthyridin-4-one), ronomilast, NVP ABE 171 (4-[8-(2,1,3-benzoxadiazol-5-yl)-1,7-naphthyridin-6-yl]benzoic acid), RPL554 (2-[(2E)-9,10-dimethoxy-4-oxo-2-(2,4,6-trimethylphenyl)imino-6,7-dihydropyrimido[6,1-a]isoquinolin-3-yl]ethylurea), CHF5480 ([(Z)-2-(3,5-dichloro-4-pyridyl)-1-(3,4-dimethoxyphenyl)vinyl](2S)-2-(4-isobutylphenyl)propanoate), or GSK256066 (6-[3-(dimethylcarbamoyl)phenyl]sulfonyl-4-(3-methoxyanilino)-8-methyl-quinoline-3-carboxamide).

In even another embodiment, the second active ingredient is a modulator of chemokine receptor function such as an antagonist of CCR1, CCR2, CCR2A, CCR2B, CCR3, CCR4, CCR5, CCR6, CCR7, CCR8, CCR9, CCR10 or CCR11 (for the C—C family), for example a CCR1, CCR2B or CCR5 receptor antagonist; CXCR1, CXCR2, CXCR3, CXCR4 or CXCR5 (for the C—X—C family), for example a CXCR2 or CXCR3 receptor antagonist; or CX₃CR1 for the C—X₃—C family. For example, the second active ingredient in one embodiment, is PS-031291 (pyrrolidine-1,2-dicarboxylic acid 2-[(4-chloro-benzyl)-methyl-amide] 1-[(4-trifluoromethyl-phenyl)-amide]), CCX-354 (1-[4-(4-chloro-3-methoxy-phenyl)piperazin-1-yl]-2-[3-(1H-imidazol-2-yl)pyrazolo[3,4-b]pyridin-1-yl]ethanone), vicriviroc, maraviroc, cenicriviroc, navarixin (2-hydroxy-N,N-dimethyl-3-[[2-[[(1R)-1-(5-methyl-2-furyl)propyl]amino]-3,4-dioxo-cyclobuten-1-yl]amino]benzamide), SB656933 (1-(2-chloro-3-fluoro-phenyl)-3-(4-chloro-2-hydroxy-3-piperazin-1-ylsulfonyl-phenyl)urea), N-[2-[(2,3-difluorophenyl)methylsulfanyl]-6-[(1R,2S)-2,3-dihydroxy-1-methyl-propoxy]pyrimidin-4-yl]azetidine-1-sulfonamide, N-[6-[(1R,2S)-2,3-dihydroxy-1-methyl-propoxy]-2-[(4-fluorophenyl)methylsulfanyl]pyrimidin-4-yl]-3-methyl-azetidine-1-sulfonamide or N-[2-[(2,3-difluorophenyl)methylsulfanyl]-6-[[(1R,2R)-2,3-dihydroxy-1-methyl-propyl]amino]pyrimidin-4-yl]azetidine-1-sulfonamide.

In another embodiment, the second active ingredient is a leukotriene biosynthesis inhibitor, 5-lipoxygenase (5-LO) inhibitor or 5-lipoxygenase activating protein (FLAP) antagonist such as TA270 (4-hydroxy-1-methyl-3-octyloxy-7-sinapinoylamino-2(1H)-quinolinone), PF-4191834 (2H-pyran-4-carboxamide, tetrahydro-4-[3-[[4-(1-methyl-1H-pyrazol-5-yl)phenyl]thio]phenyl]-), setileuton, CMI977 (1-[4-[(2S,5S)-5-[(4-fluorophenoxy)methyl]tetrahydrofuran-2-yl]but-3-ynyl]-1-hydroxy-urea), fiboflapon (3-[3-tert-butylsulfanyl-1-[[4-(6-ethoxy-3-pyridyl)phenyl]methyl]-5-[(5-methyl-2-pyridyl)methoxy]indol-2-yl]-2,2-dimethyl-propanoic acid), GSK2190915 (1H-indole-2-propanoic acid, 3-[(1,1-dimethylethyl)thio]-1-[[4-(6-methoxy-3-pyridinyl)phenyl]methyl]-α,α-dimethyl-5-[(2-pyridinyl)methoxy]-), licofelone, quiflapon (3-[3-tert-butylsulfanyl-1-[(4-chlorophenyl)methyl]-5-(2-quinolylmethoxy)indol-2-yl]-2,2-dimethyl-propanoic acid), veliflapon ((2R)-2-cyclopentyl-2-[4-(2-quinolylmethoxy)phenyl]acetic acid), ABT080 (4,4-bis[4-(2-quinolylmethoxy)phenyl]pentanoic acid), zileuton, zafirlukast, or montelukast.

In yet another embodiment, the second active ingredient is a CRTh2 antagonist or a DP2 antagonist such as ACT129968 (2-[2-[(5-acetyl-2-methoxy-phenyl)methylsulfanyl]-5-fluoro-benzimidazol-1-yl]acetic acid), AMG853 (2-[4-[4-(tert-butylcarbamoyl)-2-[(2-chloro-4-cyclopropyl-phenyl)sulfonylamino]phenoxy]-5-chloro-2-fluoro-phenyl]acetic acid), AM211 (2-[3-[2-[[benzylcarbamoyl(ethyl)amino]methyl]-4-(trifluoromethyl)phenyl]-4-methoxy-phenyl]acetic acid), 2-[4-acetamido-3-(4-chlorophenyl)sulfanyl-2-methyl-indol-1-yl]acetic acid, (2S)-2-[4-chloro-2-(2-chloro-4-ethylsulfonyl-phenoxy)phenoxy]propanoic acid, 2-[4-chloro-2-[2-fluoro-4-(4-fluorophenyl)sulfonyl-phenyl]phenoxy]acetic acid, or (2S)-2-[2-[3-chloro-4-(2,2-dimethylpyrrolidine-1-carbonyl)phenyl]-4-fluoro-phenoxy]propanoic acid.

A myeloperoxidase antagonist such as resveratrol, piceatannol, or 1-(2-isopropoxyethyl)-2-thioxo-5H-pyrrolo[3,2-d]pyrimidin-4-one, in another embodiment, is the second active ingredient, in a combination therapy embodiment.

In yet another combination therapy embodiment, the second active ingredient is a toll-like receptor agonist (such as a TLR7 or TLR9 agonist); an adenosine antagonist; a glucocorticoid receptor agonist (steroidal or non-steroidal); a p38 antagonist; a PDE4 antagonist; a modulator of chemokine receptor function (such as a CCR1, CCR2B, CCR5, CXCR2 or CXCR3 receptor antagonist); and/or a CRTh2 antagonist;

In one combination therapy embodiment, the compound of the disclosure, or a pharmaceutically acceptable salt thereof, is administered concurrently or sequentially with one or more further active ingredients selected from one or more of those provided above. For example, the compound of Formula (I), or a pharmaceutically acceptable salt thereof, may be administered concurrently or sequentially with a further pharmaceutical composition for use as a medicament for the treatment of bronchiectasis, e.g., non-CF bronchiectasis. Said further pharmaceutical composition may be a medicament which the patient may already be prescribed (e.g. an existing standard or care medication), and may itself be a composition comprising one or more active ingredients selected from those defined above.

The dosage administered will vary with the compound employed, the mode of administration, the treatment desired and the disorder indicated. For example, in one embodiment, the daily dosage of the compound of Formula (I), if inhaled, may be in the range from 0.05 micrograms per kilogram body weight (μg/kg) to 100 micrograms per kilogram body weight (μg/kg). Alternatively, in one embodiment, if the compound is administered orally, then the daily dosage of the compound of the disclosure may be in the range from 0.01 micrograms per kilogram body weight (μg/kg) to 100 milligrams per kilogram body weight (mg/kg).

In one embodiment, the compound of formula (I) is administered in an oral dosage form. In a further embodiment, the compound of formula (I) is administered as a 10 mg to 50 mg dosage form, for example, a 10 mg dosage form, a 15 mg dosage form, a 20 mg dosage form, a 25 mg dosage form, a 30 mg dosage form or a 50 mg dosage form. In a further embodiment, the dosage form is 10 mg or 25 mg. In a further embodiment, the dosage form is administered once daily. In even a further embodiment, the compound is (2S)—N-{(1S)-1-cyano-2-[4-(3-methyl-2-oxo-2,3-dihydro-1,3-benzoxazol-5-yl)phenyl]ethyl}-1,4-oxazepane-2-carboxamide, or a pharmaceutically acceptable salt thereof.

The compounds of formula (I), or pharmaceutically acceptable salts thereof, may be used on their own but will generally be administered in the form of a pharmaceutical composition in which the formula (I) compound/salt (active ingredient) is in a composition comprising a pharmaceutically acceptable adjuvant(s), diluents(s) and/or carrier(s). Conventional procedures for the selection and preparation of suitable pharmaceutical formulations are described in, for example, “Pharmaceuticals—The Science of Dosage Form Designs”, M. E. Aulton, Churchill Livingstone, 2^{nd} Ed. 2002, incorporated by reference herein in its entirety for all purposes.

Depending on the mode of administration, the pharmaceutical composition will comprise from 0.05 to 99% w (percent by weight), for example, from 0.05 to 80% w, or from 0.10 to 70% w, or from 0.10 to 50% w, of active ingredient, all percentages by weight being based on total composition.

In one oral administration embodiment, the oral dosage form is a film-coated oral tablet. In a further embodiment, the dosage form is an immediate release dosage form with rapid dissolution characteristics under in vitro test conditions.

In one embodiment, the oral dosage form is administered once daily. In a further embodiment, the oral dosage form is administered at approximately the same time every day, e.g., prior to breakfast. In another embodiment, the composition comprising an effective amount of formula (I) is administered 2× day. In yet another embodiment, the composition comprising an effective amount of formula (I) is administered 1× week, 2× week, 3× week, 4× week, or 5× week.

For oral administration the compound of the disclosure may be admixed with adjuvant(s), diluent(s) or carrier(s), for example, lactose, saccharose, sorbitol, mannitol; starch, for example, potato starch, corn starch or amylopectin; cellulose derivative; binder, for example, gelatine or polyvinylpyrrolidone; disintegrant, for example cellulose derivative, and/or lubricant, for example, magnesium stearate, calcium stearate, polyethylene glycol, wax, paraffin, and the like, and then compressed into tablets. If coated tablets are required, the cores, prepared as described above, may be coated with a suitable polymer dissolved or dispersed in water or readily volatile organic solvent(s). Alternatively, the tablet may be coated with a concentrated sugar solution which may contain, for example, gum arabic, gelatine, talcum and titanium dioxide.

For the preparation of soft gelatine capsules, the compound of the disclosure may be admixed with, for example, a vegetable oil or polyethylene glycol. Hard gelatine capsules may contain granules of the compound using pharmaceutical excipients like the above-mentioned excipients for tablets. Also liquid or semisolid formulations of the compound of the disclosure may be filled into hard gelatine capsules.

In one embodiment, the composition is an oral disintegrating tablet (ODT). ODTs differ from traditional tablets in that they are designed to be dissolved on the tongue rather than swallowed whole

In one embodiment, the composition is an oral thin film or an oral disintegrating film (ODF). Such formulations, when placed on the tongue, hydrate via interaction with saliva, and releases the active compound from the dosage form. The ODF, in one embodiment, contains a film-forming polymer such as hydroxypropylmethylcellulose (HPMC), hydroxypropyl cellulose (HPC), pullulan, carboxymethyl cellulose (CMC), pectin, starch, polyvinyl acetate (PVA) or sodium alginate.

Liquid preparations for oral application may be in the form of syrups, solutions or suspensions. Solutions, for example may contain the compound of the disclosure, the balance being sugar and a mixture of ethanol, water, glycerol and propylene glycol. Optionally such liquid preparations may contain coloring agents, flavoring agents, saccharine and/or carboxymethylcellulose as a thickening agent. Furthermore, other excipients known to those skilled in art may be used when making formulations for oral use.

The skilled person will recognise that the compounds of the disclosure may be prepared, in known manner, in a variety of ways. The routes below are merely illustrative of some of the methods that can be employed for the synthesis of compounds of formula (I).

The present disclosure further provides a process for the preparation of a compound of formula (I) or a pharmaceutically acceptable salt thereof as defined above which comprises reacting a compound of formula (II),

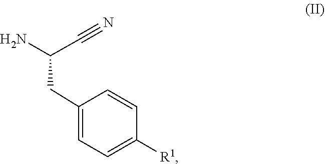

wherein R¹ is as defined in formula (I), with a compound of formula (III),

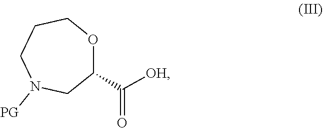

wherein PG represents a protecting group (e.g. tert-butoxycarbonyl), and optionally thereafter carrying out one or more of the following procedures:
converting a compound of formula (I) into another compound of formula (I);
removing any protecting groups; and/or
forming a pharmaceutically acceptable salt.

The process is conveniently carried out in the presence of a base such as DiPEA or TEA and one or more activating agents such as EDCI, 2-pyridinol-1-oxide, or T3P. The reaction is conveniently carried out in an organic solvent such as DMF or DCM at a temperature, for example, in the range from 20° C. to 100° C., in particular at ambient temperature (25° C.).

Compounds of formula (II) may be prepared by reaction of a compound of formula (IV),

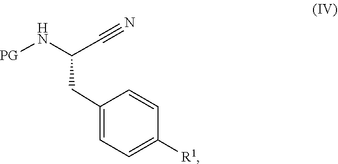

wherein PG represents a protecting group (e.g. tert-butoxycarbonyl), with a suitable reagent to remove the protecting group PG. An example of a suitable reagent is formic acid.

Compounds of formula (IV) may be prepared by reacting a compound of formula (V),

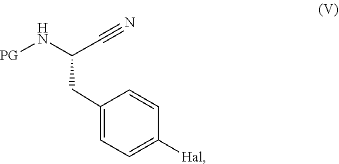

wherein PG represents a protecting group (e.g., tert-butoxycarbonyl) and Hal represents a halogen (e.g. I or Br), with a compound of formula (VI) or an ester thereof,

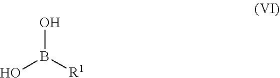

wherein R¹ is as defined in formula (I), in the presence of a catalyst such as Pd(dppf)Cl₂ DCM or 1,1 bis(di-tert-butylphosphino)ferrocene palladium dichloride and a base such as potassium carbonate or sodium carbonate. The reaction is conveniently carried out in a solvent such as dioxane/water mixture or ACN/water mixture at a temperature, for example, in the range from 20° C. to 100° C., particularly at 75° C.

Compounds of formula (V) may be prepared from a compound of formula (VII),

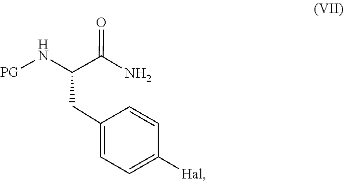

in which PG represents a protecting group (e.g. tert-butoxycarbonyl) and Hal represents a halogen (e.g., I or Br), using standard literature procedures for the dehydration of an amide, for example with Burgess reagent, or with a reagent such as T3P with or without a base such as DiPEA, in a solvent such as DCM or DMF at a temperature in the range from −20° C. to 100° C., for example at 0° C.

Compounds of formula (VII) may be prepared by reacting a compound of formula (VIII),

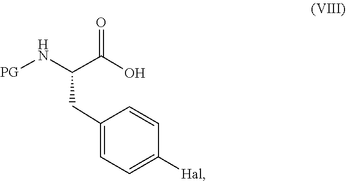

in which PG represents a protecting group (e.g. tert-butoxycarbonyl) and Hal represents a halogen (e.g., I or Br), with an aqueous ammonia solution, using standard literature procedures for the formation of an amide, for example, in the presence of a base such as N-ethyl-morpholine or DiPEA and an activating agent such as TBTU or T3P. The reaction is conveniently carried out in an organic solvent such as DMF, at a temperature in the range from −20° C. to 100° C., for example at 0° C.

Compounds of formula (VIII) are either commercially available, are known in the literature (e.g., from Tetrahedron: Asymmetry, 1998, 9, 503, incorporated by reference herein in its entirety for all purposes) or may be prepared using known techniques.

There is further provided a process for the preparation of a compound of formula (I), or a pharmaceutically acceptable salt thereof, as defined above which comprises reacting a compound

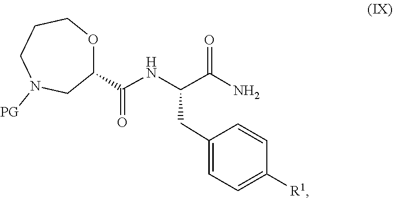

wherein R¹ is as defined above and PG represents a protecting group (e.g. tert-butoxycarbonyl), using standard literature procedures for the dehydration of an amide, for example with Burgess reagent or with a reagent such as T3P with or without a base such as DiPEA, in a solvent such as DCM or DMF at a temperature in the range from −20° C. to 100° C., for example at 25° C., and thereafter reacting with a suitable reagent to remove the protecting group PG. An example of a suitable reagent is formic acid.

A compound of formula (IX) may be prepared by reacting a compound of formula (X), wherein PG represents a protecting group (e.g. tert-butoxycarbonyl),

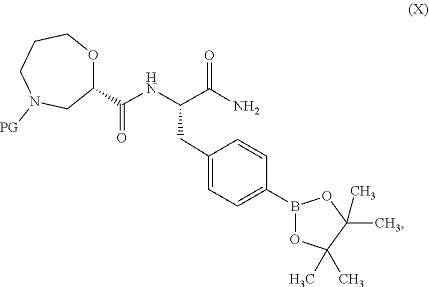

with a halide of formula (XI), wherein R¹ is defined as in formula (I), R¹—Br/I (XI), in the presence of a catalyst such as bis[bis(1,2-diphenylphosphino)ethane]palladium(0), or Pd(dppf)Cl₂ DCM, and a base such as potassium carbonate or sodium carbonate. The reaction is conveniently carried out in a solvent such as dioxane/water mixture or ACN/water mixture at a temperature, for example, in the range from 20° C. to 100° C., particularly at 80° C.

A compound of formula (X) may be prepared by reacting a compound of formula (XII), wherein PG represents a protecting group (e.g. tert-butoxycarbonyl),

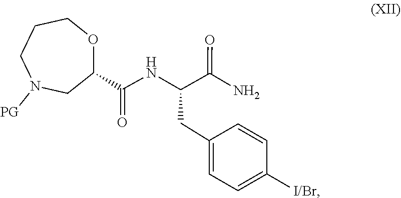

with B₂Pin₂ in the presence of a suitable catalyst such as Pd(dppf)Cl₂·DCM and with or without 1,1′-bis(diphenylphosphino)ferrocene or 1,1-bis(di-tert-butylphosphino)ferrocene palladium dichloride, with a suitable salt such as potassium acetate, in a solvent such as DMSO at a temperature in the range 60° C. to 100° C., for example at 85° C.

A compound of formula (XII) may be prepared by reacting a compound of formula (XIII),

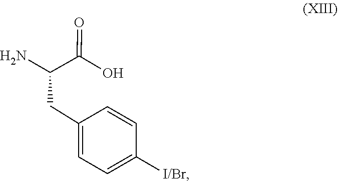

with a compound of formula (III),

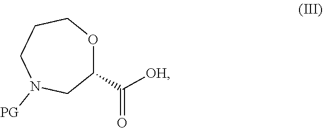

wherein PG represents a protecting group (e.g. tert-butoxycarbonyl) in the presence of a base such as DiPEA or TEA and an activating agent such as EDCI, 2-pyridinol-1-oxide, or T3P. The reaction is conveniently carried out in an organic solvent such as DMF or DCM at a temperature, for example, in the range from 20° C. to 100° C., in particular at ambient temperature (25° C.).

Compounds of formula (XIII) may be prepared by reacting a compound of formula (XIV),

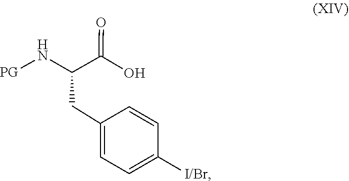

in which PG is as defined in formula (VII), with an aqueous ammonia solution, using standard literature procedures for the formation of an amide, for example, in the presence of a base such as N-ethyl-morpholine or DiPEA and an activating agent such as a “uronium” reagent (for example TBTU), or T3P. The reaction is conveniently carried out in an organic solvent such as DMF, at a temperature in the range from −20° C. to 100° C., for example at 0° C.

A compound of formula (IX) may be prepared by reacting a compound of formula (XII) wherein PG represents a protecting group (e.g. tert-butoxycarbonyl), with a compound of formula (VI) or a boronate ester thereof, in the presence of a catalyst such as bis[bis(1,2-diphenylphosphino)ethane]palladium(0) or Pd(dppf)Cl₂·DCM and a base such as potassium carbonate or sodium carbonate. The reaction is conveniently carried out in a solvent such as dioxane/water or ACN/water mixture at a temperature, for example, in the range from 20° C. to 100° C., particularly at 80° C.

There is further provided a process for the preparation of a compound of formula (I), or a pharmaceutically acceptable salt thereof, as defined above which comprises reacting a compound of formula (XV),

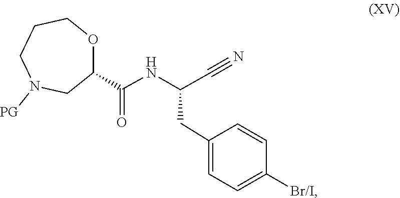

wherein PG represents a protecting group (e.g. tert-butoxycarbonyl), with a compound of formula (VI) or an ester thereof, wherein R¹ is as defined in formula (I), in the presence of a catalyst such as Pd(dppf)Cl₂·DCM or 1,1 bis(di-tert-butylphosphino)ferrocene palladium dichloride and a base such as potassium carbonate or sodium carbonate. The reaction is conveniently carried out in a solvent such as dioxane/water mixture or ACN/water mixture at a temperature, for example, in the range from 20° C. to 100° C., particularly at 75° C., and thereafter reacting with a suitable reagent to remove the protecting group PG. An example of a suitable reagent is formic acid.

Compounds of formula (XV) may be prepared from compounds of formula (XII) using standard procedures for the dehydration of an amide, for example with Burgess reagent or a reagent such as TBTU or T3P with or without a base such as DiPEA, in a solvent such as DCM or DMF at a temperature in the range from −20° C. to 100° C., for example at 25° C.

There is further provided a process for the preparation of a compound of formula (I) or a pharmaceutically acceptable salt thereof as defined above which comprises reacting a compound of formula (XVI),

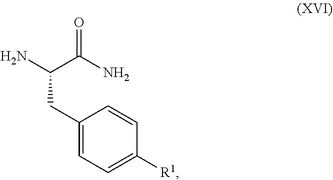

wherein R¹ is as defined in formula (I), with a compound of formula (III), conveniently carried out in the presence of a base such as DiPEA or TEA and one or more activating agents such as EDCI, 2-pyridinol-1-oxide, or T3P, followed by a dehydrating reagent such as T3P. The reaction is conveniently carried out in an organic solvent such as DMF or DCM at a temperature, for example, in the range from 20° C. to 100° C., in particular at ambient temperature (25° C.).

Compounds of formula (XVI) can be prepared from reacting compounds of formula (VII) with compounds of formula (VI) or an ester thereof, wherein R¹ is as defined in formula (I), in the presence of a catalyst such as Pd(dppf)Cl₂·DCM or 1,1 bis(di-tert-butylphosphino)ferrocene palladium dichloride and a base such as potassium carbonate or sodium carbonate. The reaction is conveniently carried out in a solvent such as dioxane/water mixture or ACN/water mixture at a temperature, for example, in the range from 20° C. to 100° C., particularly at 75° C., followed by deprotection of PG.

A compound of formula (III),

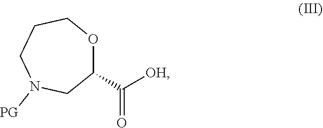

wherein PG represents a protecting group (e.g. tert-butoxycarbonyl) is either commercially available, or may be prepared from a compound of formula (XVII),

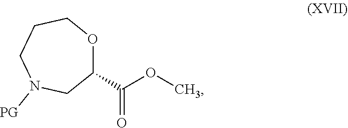

using literature procedures for mild ester hydrolysis (e.g. from Ter. Lett., 2007, 48, 2497, incorporated by reference herein in its entirety for all purposes), for example with LiBr and a base such as TEA, in a solvent such as ACN/water mixture, for example at 25° C.

A compound of formula (XVII), wherein PG represents a protecting group (e.g. tert-butoxycarbonyl), may be prepared from a compound of formula (XVIII),

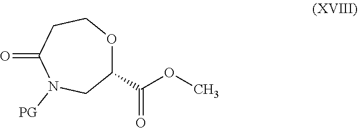

using a reducing agent, for example BH₃-DMS, in a solvent such as THF, at a temperature in the range from 0 to 40° C., for example at 25° C.

A compound of formula (XVII), where PG represents a protecting group (e.g. tert-butoxycarbonyl), may be prepared from a compound of formula (XIX), using a biocatalytic transformation for chemoselective lactam formation, e.g., using a lipase such as Novozym 435, in a solvent such as an ether, e.g., dioxane, at a temperature in the range from 0 to 80° C., for example at 55° C., followed by conditions for introduction of the protecting group PG.

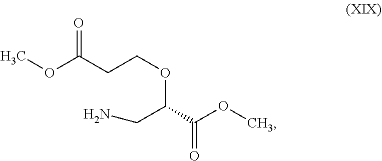

A compound of formula (XIX) may be prepared from a compound of formula (XX),

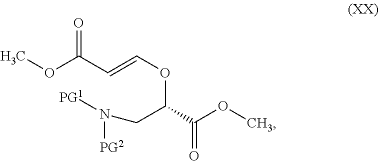

wherein PG¹ and PG² are protecting groups (e.g., benzyl), using conditions for hydrogenation, for example using H₂ (g), and a reagent such as palladium dihydroxide on carbon, in a solvent such as methanol or dioxane, under a pressure of for example 10 bar, at a temperature in the range from 25 to 80° C., for example at 40° C.

A compound of formula (XX), wherein PG¹ and PG² are protecting groups (e.g., benzyl), may be prepared from a compound of formula (XXI),

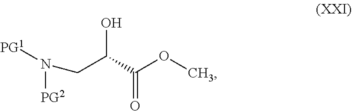

wherein PG¹ and PG² are protecting groups (e.g. benzyl), using conditions for Oxa-Michael reaction, reacting with methyl propynoate, in presence of a base such as 4-methylmorpholine, in a solvent such as toluene, at a temperature in the range from 0 to 100° C., for example at 25° C.

A compound of formula (XXI), w wherein PG¹ and PG² are protecting groups (e.g. benzyl), may be prepared from reacting a diprotected benzyl amine (e.g., dibenzylamine) with (S)-methyl oxirane-2-carboxylate, in a solvent such as ethanol, at a temperature in the range from 0 to 78° C., for example at 70° C.

Alternatively, a compound of formula (III),

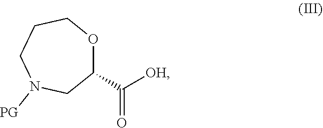

wherein PG represents a protecting group (e.g. tert-butoxycarbonyl) may be prepared from oxidation of a compound of formula (XXII),

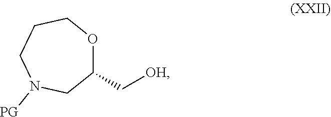

for example, using reagents such as TEMPO, and sodium hypochlorite, optionally in presence of a salt such as sodium bromide, in a solvent such as DCM/water, and in presence of a buffer such as NaHCO₃, and a phase transfer catalyst such as tetrabutylammonium bisulphate, at a temperature in the range from 0 to 100° C., e.g., at 25° C.

A compound of formula (XXII), wherein PG represents a protecting group (e.g., tert-butoxycarbonyl) may be prepared from a compound of formula (XXIII),

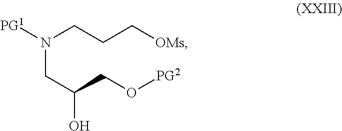

wherein PG¹ and PG² are protecting groups (e.g. benzyl), reacting with a base such as sodium hydride, in a solvent such as THF, at a temperature in the range from 0 to 60° C., e.g., 25° C., followed by interconversion of protecting groups PG, PG¹ and PG², as defined in formula (XXII) and (XXIII).

A compound of formula (XXII), wherein PG¹ and PG² are protecting groups (e.g., benzyl), may be prepared from reacting protected 3-aminopropanol (e.g. N-benzyl-3-aminopropanol) with (S)-2-((benzyloxy)methyl)oxirane, in a solvent such as ethanol or propanol, at a temperature in the range from 0 to 70° C., for example at 40° C., followed by reacting the crude product with methanesulfonyl chloride, in presence of a base such as DiPEA, in a solvent such as DCM, at a temperature in the range from −10 to 25° C., e.g., −5° C.

Compounds of formula (VI) or an ester thereof, (VIII), (XI) and (XIV) are either commercially available, are known in the literature or may be prepared using known techniques.

It will be appreciated by those skilled in the art that in the processes of the present disclosure certain functional groups such as hydroxyl or amino groups in the reagents may need to be protected by protecting groups. Thus, the preparation of the compounds of formula (I) may involve, at an appropriate stage, the removal of one or more protecting groups.

The skilled person will recognise that at any stage of the preparation of the compounds of formula (I), mixtures of isomers (e.g., racemates) of compounds corresponding to any of formulae (II)-(V), (VII)-(X) and (XXII)-(XVI) may be utilized. At any stage of the preparation, a single stereoisomer may be obtained by isolating it from a mixture of isomers (e.g., a racemate) using, for example, chiral chromatographic separation.

The protection and deprotection of functional groups is described in ‘Protective Groups in Organic Synthesis’, 4^{th} Ed, T. W. Greene and P. G. M. Wuts, Wiley (2006) and ‘Protecting Groups’, 3^{rd} Ed P. J. Kocienski, Georg Thieme Verlag (2005), incorporated by reference herein in its entirety for all purposes.

As provided throughout, according to the methods provided herein, a compound of formula (I) can be administered as a pharmaceutically acceptable salt. A pharmaceutically acceptable salt of a compound of formula (I) may be advantageous due to one or more of its chemical or physical properties, such as stability in differing temperatures and humidities, or a desirable solubility in H₂O, oil, or other solvent. In some instances, a salt may be used to aid in the isolation or purification of the compound of formula (I).

Where the compound of formula (I) is sufficiently acidic, pharmaceutically acceptable salts include, but are not limited to, an alkali metal salt, e.g., Na or K, an alkali earth metal salt, e.g., Ca or Mg, or an organic amine salt. Where the compound of formula (I) is sufficiently basic, pharmaceutically acceptable salts include, but are not limited to, inorganic or organic acid addition salts.

There may be more than one cation or anion depending on the number of charged functions and the valency of the cations or anions.

For reviews on suitable salts, and pharmaceutically acceptable salts amenable for use herein, see Berge et al., J. Pharm. Sci., 1977, 66, 1-19 or “Handbook of Pharmaceutical Salts: Properties, selection and use”, P. H. Stahl, P. G. Vermuth, IUPAC, Wiley-VCH, 2002, incorporated by reference herein in its entirety for all purposes.

The compounds of formula (I) may form mixtures of its salt and co-crystal forms. It is also to be understood that the methods provided herein can employ such salt/co-crystal mixtures of the compound of formula (I).

Salts and co-crystals may be characterized using well known techniques, for example X-ray powder diffraction, single crystal X-ray diffraction (for example to evaluate proton position, bond lengths or bond angles), solid state NMR, (to evaluate for example, C, N or P chemical shifts) or spectroscopic techniques (to measure for example, O—H, N—H or COOH signals and IR peak shifts resulting from hydrogen bonding).

It is also to be understood that certain compounds of formula (I) may exist in solvated form, e.g., hydrates, including solvates of a pharmaceutically acceptable salt of a compound of formula (I).

In one embodiment, certain compounds of formula (I) may exist as racemates and racemic mixtures, single enantiomers, individual diastereomers and diastereomeric mixtures. It is to be understood that the present disclosure encompasses all such isomeric forms. Certain compounds of formula (I) may also contain linkages (e.g., carbon-carbon bonds, carbon-nitrogen bonds such as amide bonds) wherein bond rotation is restricted about that particular linkage, e.g. restriction resulting from the presence of a ring bond or double bond. Accordingly, it is to be understood that the methods provided herein can employ such isomers. Certain compound of formula (I) may also contain multiple tautomeric forms. It is to be understood that the present disclosure encompasses all such tautomeric forms. Stereoisomers may be separated using conventional techniques, e.g. chromatography or fractional crystallization, or the stereoisomers may be made by stereoselective synthesis.

In a further embodiment, the compounds of formula (I) encompass any isotopically-labeled (or “radio-labelled”) derivatives of a compound of formula (I). Such a derivative is a derivative of a compound of formula (I) wherein one or more atoms are replaced by an atom having an atomic mass or mass number different from the atomic mass or mass number typically found in nature. Examples of radionuclides that may be incorporated include ²H (also written as “D” for deuterium). As such, in one embodiment, a compound of formula (I) is provided where one or more hydrogen atoms are replaced by one or more deuterium atoms; and the deuterated compound is used in one of the methods provided herein for treating bronchiectasis. In a further embodiment, the bronchiectasis is non-CF bronchiectasis.

In a further embodiment, the compounds of formula (I) may be administered in the form of a prodrug which is broken down in the human or animal body to give a compound of the formula (I). Examples of prodrugs include in vivo hydrolysable esters of a compound of the formula (I).

An in vivo hydrolysable (or cleavable) ester of a compound of the formula (I) that contains a carboxy or a hydroxy group is, for example, a pharmaceutically acceptable ester which is hydrolyzed in the human or animal body to produce the parent acid or alcohol. For examples of ester prodrugs derivatives, see: Curr. Drug. Metab. 2003, 4, 461, incorporated by reference herein in its entirety for all purposes.

Various other forms of prodrugs are known in the art, and can be used in the methods provided herein. For examples of prodrug derivatives, see: Nature Reviews Drug Discovery 2008, 7, 255, the disclosure of which is incorporated by reference herein in its entirety for all purposes.

### Example

The present invention is further illustrated by reference to the following Example. However, it should be noted that this Example, like the embodiments described above, are illustrative and are not to be construed as restricting the scope of the invention in any way.

### Example—Efficacy. Safety and Tolerability, and Pharmacokinetics of (2S)—N-{(1S)-1-cyano-2-[4-(3-methyl-2-oxo-2,3-dihydro-1,3-benzoxazol-5-yl) phenyl]ethyl}-1,4-oxazepane-2-carboxamide Administered Once Daily for 24 Weeks in Subjects with Non-Cystic Fibrosis Bronchiectasis

The efficacy of (2S)—N-{(1S)-1-cyano-2-[4-(3-methyl-2-oxo-2,3-dihydro-1,3-benzoxazol-5-yl)phenyl]ethyl}-1,4-oxazepane-2-carboxamide

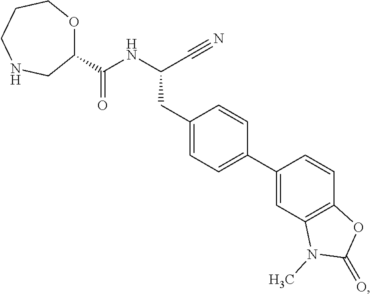 [SMILES: Cn1c(=O)oc2ccc(-c3ccc(C[C@@H](C#N)NC(=O)[C@@H]4CNCCCO4)cc3)cc21]

referred to in this example as “INS1007”, administered once daily (QD) for 24 weeks in subjects with non-cystic fibrosis (CF) bronchiectasis (NCFBE) is assessed. Subjects are randomized in a 1:1:1 ratio to 3 treatment arms to receive either (i) 10 mg INS1007; (ii) 25 mg INS1007 or (iii) matching placebo.

Following a screening visit (Visit 1) and a screening period of up to 4 weeks, subjects are randomized at Visit 2 (Day 1, “Baseline”) and return thereafter for study visits at 2 weeks (Visit 3), 4 weeks (Visit 4), 8 weeks (Visit 5), 12 weeks (Visit 6), 16 weeks (Visit 7), 20 weeks (Visit 8), 24 weeks (Visit 9) and 28 weeks (Visit 10). During each visit, assessments and procedures are performed to enable the evaluation of the criteria described below. Study treatment occurs between Visits 2-9.

At Week 28 (Visit 10), blood and sputum samples are collected for biomarker assessment.

The time to the first pulmonary exacerbation over the 24-week treatment period will be assessed.

The following additional criteria will be assessed.
1. Change from Baseline in Quality of Life-Bronchiectasis (QOL-B) Respiratory Symptoms Domain score over the 24-week treatment period.
2. Change from Screening in post-bronchodilator FEV₁ over the 24-week treatment period.
3. Change in concentration of active neutrophil elastase (NE) in sputum from pre-treatment (defined as the average of Screening and Day 1 concentrations) to on-treatment (defined as the average of Week 12 and Week 24 concentrations).
4. Rate of pulmonary exacerbations (number of events per person/time) over the 24-week treatment period.
5. Change from Baseline in QOL-B scores (all domains excluding the Respiratory Symptoms Domain) over the 24-week treatment period.
6. Change from Baseline in Leicester Cough Questionnaire (LCQ) score over the 24-week treatment period. See, e.g., Murray et al. (2003). Thorax 58(4), pp. 339-343, incorporated by reference herein in its entirety.
7. Change from Baseline in St. George's Respiratory Questionnaire (SGRQ) total score over the 24-week treatment period.
8. Change in active NE concentration in sputum from pre-treatment to Weeks 2, 4, and 28.
9. Change in concentration of active NE in reagent-stimulated blood from pre-treatment to Weeks 2, 4, 12, 24, and 28.
10. Change from Baseline in sputum color (assessed by the sputum color chart) at Weeks 2, 4, 12, 24, and 28.
11. Change from Baseline in desmosine in urine at Weeks 2, 4, 12, 24, and 28.
12. Change from Screening of forced vital capacity (FVC) at Weeks 12 and 24.
13. Change from Screening of peak expiratory flow rate (PEFR) at Weeks 12 and 24.
14. Change from Screening of forced expiratory flow 25%-75% (FEF₂₅₋₇₅) at Weeks 12 and 24.
15. Total duration (in days) of exacerbations, per subject, over the 24-week treatment period.
16. Frequency of use of rescue medications over the 24-week treatment period. Rescue medications include short-acting beta agonists (SABAs), short-acting muscarinic antagonists (SAMAs), newly prescribed long-acting beta agonists (LABAs), long-acting muscarinic antagonists (LAMAs), and oxygen.
17. Number of subjects hospitalized due to bronchiectasis exacerbations by the end of the 24-week treatment period.

### Pulmonary Function Test (PFT)

Pulmonary function test (PFT) by spirometry (FEV₁, FVC, PEFR, and FEF₂₅₋₇₅) will be performed per the American Thoracic Society (ATS/European Respiratory Society [ERS]) criteria at Visit 1 (Screening), Visit 6, and Visit 9. Spirometry criteria are described in Miller et al. (2005). Standardization of Spirometry. Eur. Respir. J. 26, pp. 319-38, incorporated by reference herein in its entirety for all purposes. The subject will be provided with the detailed instruction on how to conduct FVC maneuver per ATS/ERS spirometry standardization before performing the test.

Subjects will be advised to withhold short-acting inhaled drugs (e.g., the β-agonist albuterol/salbutamol or the anticholinergic agent ipratropium bromide) within 6 hr. prior to the test. Long-acting β-agonist bronchodilators (e.g., salmeterol or formoterol) or long-acting muscarinic bronchodilators (e.g., tiotropium) or oral therapy with aminophylline or slow release β-agonists should be withheld for 12-24 hours depending on the medication used for the minimum time intervals for a list of restricted medications) prior to the testing.

Subjects will be advised to withhold the use of their inhaled corticosteroids at least 24 hours prior to the test. In the event a subject has taken a restricted medication during the specified time interval before the test, the test will be rescheduled for another visit within the protocol-specified visit window. If rescheduling the visit is not feasible for the subject, the test will be conducted as usual with appropriate notation in the source documents.

### Sputum Collection

If the patient cannot produce a sputum sample on his or her own, the following procedure is used. The induction procedure starts by subject nebulization of a saline solution. The amount of saline, e.g., 3% or 7% will be decided based on the Investigator's preference. Approximately 3-6 mL of the selected saline is placed in the nebulizer, and the subject is in a sitting up or in a semi-fowler position. The subject may wear a nose clip during the nebulization. The subject will breathe slowly and deeply through the nebulizer mouthpiece inhaling the salt water mist. The subject is reminded to not breathe quickly but to have slow, deep breaths pausing at peak inspiration to allow deposition of particles. The nebulization time is 10 minutes.

At the end of nebulization, the subject is instructed to take a few deep breaths, swallow the extra saliva in his/her mouth and attempt to cough up a sputum sample. The subject is encouraged to cough forcefully using the deep coughing method and/or “huffing” cough method. All sputum is deposited in the specimen container. The container is not opened until the specimen is ready to be deposited. The container is closed immediately after depositing the sample.

The sputum sample should be approximately 3 mL—slightly below the bottom line (5 mL) on the collection container. If a sufficient sputum sample is not collected and the subject appears to be tolerating the induction procedure well, the subject can complete another 10-minute nebulization period. If a second 10-minute nebulization period is required, the recommendation is to increase the sodium chloride concentration (i.e., if 3% was used first then 7% should be used for the subsequent nebulization; if 7% was used first then 10% should be used for the subsequent nebulization). Upon completion, the sputum sample is refrigerated until it is sent to the microbiology laboratory for further analysis.

### Bronchiectasis Severity Index (BSI)

The BSI score will be calculated at Baseline as described in Table 1, below.

TABLE 1
Calculation of Bronchiectais Severity Index

| Severity criteria | 0 Point | 1 Point | 2 Point | 3 Point | 4 Point | 5 Point | 6 Point |
|---|---|---|---|---|---|---|---|
| Age | <50 | | 50-69 | — | 70-79 | — | 80+ |
| BMI kg/m2 | >18.5 | | <18.5 | — | — | — | — |
| FEV1 % | >80% | 50-80% | 30-49% | <30% | — | — | — |
| predicted | | | | | | | |
| Hospital | No | | | | | Yes | |
| admissions in | | | | | | | |
| the past 2 years | | | | | | | |
| Exacerbation | 0-2 | | 3 or More | | | | |
| frequency in | | | | | | | |
| last 12 months | | | | | | | |
| MRC | 1-3 | | 4 | 5 | | | |
| dyspnoea | | | | | | | |
| score | | | | | | | |
| Colonization | Not | Chronic | | P. aeruginosa | | | |
| status | Colonized | Colonization | | colonization | | | |
| Radiological | <3 Lobes | 3 or More Lobes | | | | | |
| severity | Involved | or Cystic Changes | | | | | |

Abbreviations-BMI = body mass index, FEV1 = forced expiratory volume in 1 second, MRC = Medical Research Council. Estimated outcomes are those observed across 5 European treatments in the original derivation and validation study
BSI FINAL SCORE:
0-4 points:
1 year outcome: 0-2.8%
4 year outcome: 0-5.3% mortality, 0-9.2% hospitalization rate
5-8 points:
1 year outcome: 0.9-4.8% mortality rate, 1-7.2% hospitalization rate
4 year outcomes: 4-11.3% mortality rate, 9.9-19.4% hospitalization rate
9+ points:
1 year outcome: 7.6-10.5% mortality rate, 16.7-52.6% hospitalization rate
4 year outcomes: 9.9-29.2% mortality rate, 41.2-80.4% hospitalization rate

All, documents, patents, patent applications, publications, product descriptions, and protocols which are cited throughout this application are incorporated herein by reference in their entireties for all purposes.

The embodiments illustrated and discussed in this specification are intended only to teach those skilled in the art the best way known to the inventors to make and use the invention.

Modifications and variation of the above-described embodiments of the invention are possible without departing from the invention, as appreciated by those skilled in the art in light of the above teachings. It is therefore understood that, within the scope of the claims and their equivalents, the invention may be practiced otherwise than as specifically described.

1. A method for treating non-cystic fibrosis (CF) bronchiectasis in a patient in need of treatment, comprising, administering to the patient once daily via oral administration a pharmaceutical composition comprising 10 mg to 50 mg of a compound of formula (I), or a pharmaceutically acceptable salt thereof, 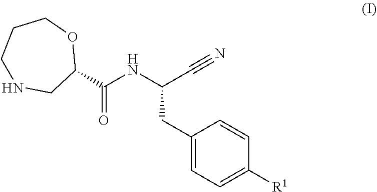
wherein,
R¹ is 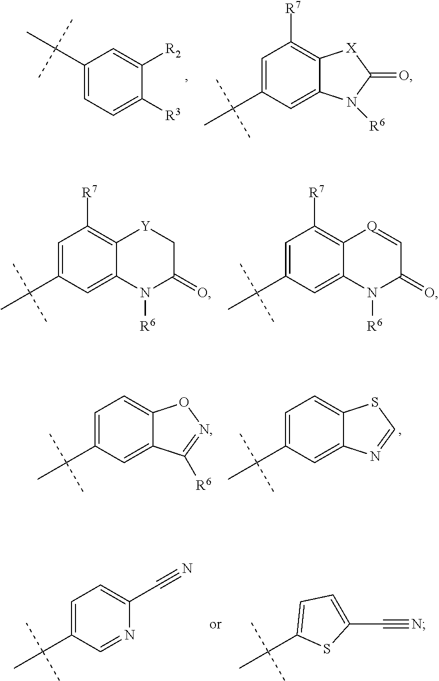
R² is hydrogen, F, Cl, Br, OSO₂C₁₋₃alkyl, or C₁₋₃alkyl;
R³ is hydrogen, F, Cl, Br, CN, CF₃, SO₂C₁₋₃alkyl, CONH₂ or SO₂NR⁴R⁵, wherein R⁴ and R⁵ together with the nitrogen atom to which they are attached form an azetidine, pyrrolidine or piperidine ring;
R⁶ is C₁₋₃alkyl, optionally substituted by 1, 2 or 3 F and/or optionally by OH, OC₁₋₃alkyl, N(C₁₋₃alkyl)₂, cyclopropyl, or tetrahydropyran;
R⁷ is hydrogen, F, Cl or CH₃;
X is O, S or CF₂;
Y is O or S; and
Q is CH or N.

2. The method of claim 1, wherein, R¹ is

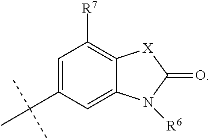

3. The method of claim 2, wherein, X is O; R⁶ is C₁₋₃alkyl; and R⁷ is hydrogen.

4. (canceled)

5. The method of claim 1, wherein the compound of Formula (I) is (2S)—N-{(1S)-1-cyano-2-[4-(3-methyl-2-oxo-2,3-dihydro-1,3-benzoxazol-5-yl)phenyl]ethyl}-1,4-oxazepane-2-carboxamide: 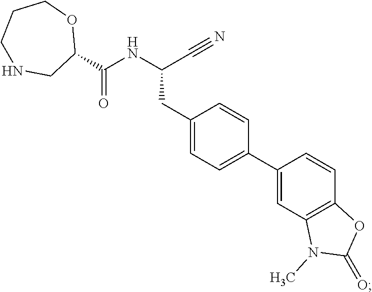 [SMILES: Cn1c(=O)oc2ccc(-c3ccc(C[C@@H](C#N)NC(=O)[C@@H]4CNCCCO4)cc3)cc21]
or a pharmaceutically acceptable salt thereof.

6. The method of claim 1, wherein the compound of Formula (I) is (2S)—N-{(1S)-1-cyano-2-[4-(3-methyl-2-oxo-2,3-dihydro-1,3-benzoxazol-5-yl)phenyl]ethyl}-1,4-oxazepane-2-carboxamide.

7. The method of claim 1, wherein the composition comprises a pharmaceutically acceptable adjuvant, diluent or carrier.

8-12. (canceled)

13. The method of claim 1, wherein the treating comprises increasing the length of time to first pulmonary exacerbation, as compared to an untreated non-CF bronchiectasis patient, wherein the pulmonary exacerbation is characterized by three or more of the following symptoms exhibited for at least 48 hours by the patient: (1) increased cough; (2) increased sputum volume or change in sputum consistency; (3) increased sputum purulence; (4) increased breathlessness and/or decreased exercise tolerance; (5) fatigue and/or malaise; (6) hemoptysis.

14. (canceled)

15. The method of claim 13, wherein the increasing comprises increasing of about 20 days to about 100 days, or from about 30 days to about 100 days, or from about 20 days to about 75 days, or from about 20 days to about 50 days, or from about 20 days to about 40 days.

16. The method of claim 1, wherein treating comprises reducing the rate of pulmonary exacerbation in the patient, as compared to the rate of pulmonary exacerbation experienced by the patient prior to treatment, or compared to an untreated non-CF bronchiectasis patient, wherein the pulmonary exacerbation is characterized by three or more of the following symptoms exhibited for at least 48 hours by the patient: (1) increased cough; (2) increased sputum volume or change in sputum consistency; (3) increased sputum purulence; (4) increased breathlessness and/or decreased exercise tolerance; (5) fatigue and/or malaise; (6) hemoptysis.

17. (canceled)

18. The method of claim 16, wherein the rate of pulmonary exacerbation in the patient is reduced by about 5%, by about 10%, by about 15%, by about 20%, by about 25%, by about 30%, by about 35%, by about 40% or by about 50%, as compared to the rate of pulmonary exacerbation experienced by the patient prior to treatment, or compared to an untreated non-CF bronchiectasis patient.

19. The method of claim 16, wherein the rate of pulmonary exacerbations in the patient is reduced by at least about 20%, as compared to the rate of pulmonary exacerbation experienced by the patient prior to treatment, or compared to an untreated non-CF bronchiectasis patient.

20-38. (canceled)

39. The method of claim 19, wherein treating comprises decreasing active neutrophil elastase (NE) sputum concentration in the patient, as compared to the active NE sputum concentration prior to treatment.

40-41. (canceled)

42. The method of claim 19, wherein treating comprises lightening the patient's sputum color as compared to the patient's sputum color prior to treatment, as measured by the sputum color chart of Murray.

43. The method of claim 42, wherein lightening the patient's sputum color comprises lightening the patient's sputum color by a single gradation.

44-59. (canceled)

60. The method of claim 1, wherein the patient is administered 10 mg of the compound of Formula (I) once daily.

61. The method of claim 1, wherein the patient is administered 25 mg of the compound of Formula (I) once daily.
